(19) United States
(12) Patent Application Publication
(10) Pub. No.: US 2023/0060967 A1
(43) Pub. Date: Mar. 2, 2023

(54) NOVEL PATHOLOGICAL MARKER AND USES THEREOF

(71) Applicant: METADEQ LIMITED, London (GB)

(72) Inventors: Lidia CASTAGNETO GISSEY, Rome (IT); Gertrude MINGRONE, Massa Martana (IT)

(73) Assignee: METADEQ LIMITED, London (GB)

(21) Appl. No.: 17/594,320

(22) PCT Filed: Apr. 8, 2020
(86) PCT No.: PCT/IB2020/053360

§ 371 Date: Oct. 11, 2021

(30) Foreign Application Priority Data
Apr. 12, 2019 (IT) 102019000005700

(51) Int. Cl. G01N 33/68; G01N 33/58

(52) U.S. Cl. CPC G01N 33/6893; G01N 33/582; G01N 2800/085

(57) ABSTRACT

The present invention relates to the identification of a novel pathological marker, novel methods for the diagnosis or for the monitoring of the progress of non-alcoholic hepatic steatosis (NAFLD) by the detection and the quantitation of said marker, and devices enabling the implementation of said methods.

The present invention relates to the identification of a novel pathological marker, novel methods for the diagnosis or for the monitoring of the progress of non-alcoholic hepatic steatosis NAFLD and/or NASH by the detection and the quantitation (quantification) of said marker, and devices enabling the implementation of said methods.

### STATE OF THE PRIOR ART

Non-alcoholic hepatic steatosis, also denoted by the acronym NAFLD (Non-Alcoholic Fatty Liver Disease) is represented by a spectrum of hepatic histological alterations characterized by an hyperaccumulation of intrahepatocyte fat (steatosis, consisting in a >5% cellular triglyceride accumulation) in the absence of significant alcohol consumption and secondary causes of hepatopathy.

NAFLD currently constitutes the main cause of alteration of hepatic cytolysis indexes in the western world among adults and, alarmingly, in children and teenagers as well.

Updated data reported in Bugianesi and Marietti 2016 (Recenti Prog Med 2016; 107: 360-368) indicate how in adult population there is a 25% NAFLD global prevalence, and how among NAFLD individuals, the global prevalence of NASH (non-alcoholic hepatic steatosis), diagnosed by liver biopsy, is of from 20 to 50%. The percentages reported herein rise dramatically in patients at risk, such as, e.g., in patients obese and/or suffering from type 2 diabetes.

The most dramatic epidemiological data concern the pediatric population, in which obesity and metabolic syndrome are in progressive global increase, by now marked also in Europe, with estimates similar to those recorded in the USA, from which there appears, from data recorded between 2007 and 2010, a prevalence of NAFLD equal to about 7%, which is a value three times higher than the values recorded less than 20 years before.

While simple steatosis possesses a negligible risk of progression to cirrhosis, a significant percentage (10-15%) of subjects with NAFLD exhibits however histological aspects of necroinflammation and ballooning degeneration characterizing the most severe form of hepatic disease, non-alcoholic steatohepatitis (NASH), which can evolve to fibrosis, cirrhosis and related complications, hepatocarcinoma (HCC) included.

It being a complex and multifactor pathology, the severity and onset of non-alcoholic hepatic steatosis are conditioned both by genetic and environmental factors. It frequently manifests itself in association with pathologies such as metabolic syndrome and type 2 diabetes, but also has a role in the syndrome onset, its development and the complications related thereto. Given NAFLD prevalence increase linked to diet and social changes of wealthy countries, since this pathology entails potential significant clinical implications, as, e.g. in a non-negligible number of cases the pathology can also evolve to non-alcoholic hepatic steatosis, —cause of hepatic cirrhosis and also of HCC—being able to identify high-risk subjects early and correctly, in order to be able to survey and predict the onset of complications that may affect the prognosis at the hepatic and extra-hepatic (cardiovascular) level is clearly important.

Besides hepatic damage, in fact, the main cause of morbility and mortality in individuals suffering from NAFLD is represented by cardiovascular complications cropping up more among these individuals than in the general population, regardless of the presence of type 2 diabetes and other risk factors.

As also reported in Bugianesi and Marietti in 2016, since NASH diagnosis is currently conditional to the performing of a liver biopsy, the efforts of the scientific community are directed, in recent years, to the search for noninvasive markers of hepatic damage applicable on a large scale and of associated genic polymorphisms, in order to correctly address the screening programs, follow-ups and the therapeutic attempts.

The identification of means for a noninvasive diagnosis enables a timely diagnosis, even in subjects that normally would not be subjected to liver biopsy, as generally a biopsy on this organ is performed only when there are serious symptoms, making it essential. Moreover, a noninvasive diagnosis can also enable to sequentially monitor the effectiveness of the therapeutic treatment performed on the patient. In fact, it should also be considered that liver biopsy under ultrasound guidance is an expensive examination that should be done in a hospital, and whose cost ranges from US$2000 to 7000 or more. Death rate by post-liver biopsy fatal hemorrhaging ranges from 0.13% to 0.33% yet can be much higher in subjects with alterations in coagulation.

Hence, the need to single out NAFLD diagnostic markers enabling an accurate and rapid diagnosis, with no need of bioptic interventions on the liver, and therefore enabling to broaden the diagnosis of the disease in the population thanks to the noninvasiveness of the diagnostic method, is currently highly felt.

### SUMMARY OF THE INVENTION

The Authors of the present invention have discovered that the Plin2 protein is an effective marker of non-alcoholic hepatic steatosis (NAFLD) and of non-alcoholic steatohepatitis (NASH) in circulating cells, finding, surprisingly, that the degree of expression of said marker measured in hepatocytes strongly and positively correlates with that measured in leukocytes of the same individual, in particular peripheral blood polymorphonuclear cells and monocytes. In addition, the Authors of the present invention have also discovered that the degree of expression of the Plin2 protein in blood cells and in hepatocytes is proportional to the NAS score in NAFLD and also to NASH severity, so that the higher the Plin2 concentration in patients suffering from NAFLD, the higher the NAS score, and the higher is the Plin2 concentration in patients suffering from NASH, the greater the severity of the disease (NAS score and NASH severity are defined according to the literature and in the present description)

The Authors of the invention have therefore devised a method for the diagnosis of NAFLD and/or NASH performed on blood samples of an individual or on leukocytes extracted from said samples in which, if Plin2 concentration in said samples is greater than that measured in one or more control samples taken from healthy individuals, the diagnosis of NAFLD and/or NASH has to be considered as ascertained.

The measuring of the degree of Plin2 expression can further be advantageously used to monitor the effectiveness of a therapy, by virtue of the correlation between said expression and the severity of the disease, whereby, the comparison between a measurement of Plin2 blood concentration of an individual performed at an instant of time t0 in which the monitoring of therapeutic effectiveness starts, enables to assess said therapeutic effectiveness, by comparing the value obtained in said measuring with that obtained in one or more subsequent measurements performed in instants of time tn, wherein n is an integer greater than 0, and wherein said instants of time are progressively subsequent (following or next) to each other and all following t0, as a decrease of Plin2 concentration over time is indicative of therapeutic effectiveness. Values constant over time indicate a containment of the disease whereas an increase over time of said values indicates a therapeutic ineffectiveness.

Moreover, the measurement in subsequent times as described above enables to monitor the course of the disease also in the absence of a pharmacological therapy, e.g. following a change of the patient's diet and life habits, of drugs or of bariatric surgery.

The invention therefore advantageously enables to perform a diagnosis and/or a monitoring of NAFLD or NASH with no need of a liver biopsy, with obvious and evident medical and economic advantages. Moreover, the simplicity of diagnosis and/or of monitoring according to the invention enables to significantly broaden the number of individuals on which the analysis can be performed, enables to easily perform also tests on children, and, moreover enables to check the state of health with regard to NAFLD or NASH also on a whole population of patients that normally would not have been subjected to liver biopsy. Furthermore, the analysis according to the present invention also enables to perform screenings on populations.

Moreover, given the greater frequency of cardiovascular damage in patients suffering from NAFLD and NASH, which proves to be independent of the presence or absence of other risk factors, an early diagnosis of the disease enables a preventive monitoring as to the cardiovascular system in said patients.

The Authors of the present invention have also discovered that the Plin2 protein concentration values, as mentioned above, correlate with the NAS score and also correlate with the severity grade of NASH, that, in the literature is defined as mild, moderate and severe on the basis of histological parameters observed in bioptic samples. Therefore, the invention also relates to methods, computer programs and devices to define the severity grade of NASH in a patient suffering from said pathology, on the basis of the Plin2 protein concentration values in biological samples of said patient as defined herein.

The Authors of the present invention have also surprisingly discovered that the values of Pnpla3 and Rab14 protein expression in the same biological samples enable to diagnose the presence of hepatic fibrosis and the severity thereof.

The method of the invention can also be carried out with a simple device enabling the aforesaid measuring (measurements), and optionally the processing of obtained data.

Therefore, object of the invention are:

a method for the diagnosis of non-alcoholic hepatic steatosis (NAFLD) and/or or of non-alcoholic steatohepatitis (NASH), comprising the steps of

a. measuring the concentration value of the Plin2 protein in a blood sample or in a sample of leukocytes extracted from said blood sample of an individual

b. comparing the value obtained in point a. with the concentration value of the Plin2 protein in a blood sample or in a sample of leukocytes extracted from said blood sample of a healthy individual,

c. diagnosing NAFLD and/or NASH when the value measured in a. is greater than the value measured in b.;

a method for the diagnosis of NAFLD or NASH, comprising the steps of

a. measuring the concentration value of the Plin2 protein in a blood sample or in a sample of leukocytes extracted from said blood sample

b. measuring the concentration value of the Plin2 protein in a population of blood samples coming from patients suffering from NAFLD and of healthy patients and identifying a cutoff value, C0, of said concentration between patients suffering from NAFLD and healthy patients, and measuring the concentration value of the Plin2 protein in a population of blood samples coming from patients suffering from NASH and of healthy patients and identifying a cutoff value, C1, of said concentration between patients suffering from NASH and healthy patients,

c. comparing the value obtained in point a. with the cutoff values obtained in point b,

d. diagnosing NAFLD when the value obtained in a. is greater than or equal to said value C0 and lower than said value C1, or diagnosing NASH when the value obtained in a. is greater than or equal to said value C1;

a method for the diagnosis of the NAS of patients suffering from NAFLD or NASH, comprising the steps of

a. measuring the concentration value of the Plin2 protein in a blood sample or in a sample of leukocytes extracted from said blood sample

b. measuring the concentration value of the Plin2 protein in a population of blood samples coming from patients suffering from NAFLD or NASH and of healthy patients wherein, in said population comprising samples coming from patients suffering from NAFLD, the NAS value was histologically defined in said patients as NAS 1, NAS 2 and NAS 3, on the basis of the percentage (percent) of steatosis in liver cells, the occurrence of ballooned hepatocytes, the presence of lobular inflammation and the presence of portal inflammation and identifying:

a cutoff value, C2, of said concentration between healthy patients and patients suffering from NAFLD with NAS 1;

a cutoff value, C3, of said concentration between patients suffering from NAFLD with NAS 1 and patients suffering from NAFLD with NAS 2; and

a cutoff value, C4, of said concentration between patients suffering from NAFLD with NAS 2 and patients suffering from NASH with NAS 3,

c. comparing the value obtained in point a. with the cutoff values obtained in point b

d. diagnosing the NAS degree as

NAS 1 when the value obtained in a. is greater than or equal to said cutoff value C2 and lower than or equal to said cutoff value C3,

NAS 2 when the value obtained in a. is greater than said value C2 and lower than or equal to said cutoff value C4,

NAS 3 when the value obtained in a. is greater than said value C4;

A method for the diagnosis of the severity grade of NASH, comprising the steps of

a. measuring the concentration value of the Plin2 protein in a blood sample or in a sample of leukocytes extracted from said blood sample

b. measuring the concentration value of the Plin2 protein in a population of blood samples coming from patients suffering from NASH and of healthy patients wherein, in said population comprising samples coming from patients suffering from NASH, the severity grade of the pathology was histologically defined in said patients as mild, moderate or severe on the basis of the percentage of steatosis in liver cells, the occurrence of ballooned hepatocytes, the presence of lobular inflammation and the presence of portal inflammation and identifying

a cutoff value, C5, of said concentration between healthy patients and patients suffering from mild NASH;

a cutoff value, C6, of said concentration between patients suffering from mild NASH and patients suffering from moderate NASH; and

a cutoff value, C7, of said concentration between patients suffering from moderate NASH and patients suffering from severe NASH,

c. comparing the value obtained in point a. with the cutoff values obtained in point b

d. diagnosing the severity grade of NASH as

mild when the value obtained in a. is greater than said value C5 and lower than said value C6,

moderate, when the value obtained in a. is greater than said value C6 and lower than said value C7, and

severe, when the value obtained in a. is greater than said value C7;

a method for the diagnosis of NAFLD or NASH, comprising the steps of

a. measuring the concentration value of the Plin2 protein in a blood sample or in a sample of leukocytes extracted from said blood sample, wherein said value is measured by cytofluorimetry using a monoclonal antibody specifically binding Plin2 and not binding other proteins labeled with a suitable fluorochrome

b. comparing the value obtained in point a. with a cutoff value of said concentration expressed in terms of mean fluorescence intensity (MFI) equal to 2.7 MFI and with a cutoff value of said concentration equal to 1.0 MFI

d. diagnosing NAFLD when the value obtained in a. is greater than or equal to 1.0 MFI and lower than or equal to 2.7 MFI, or diagnosing NASH when the value obtained in a. is greater than 2.7 MFI;

a method for the diagnosis of the NAS of patients suffering from NAFLD or NASH, comprising the steps of

a. measuring the concentration value of the Plin2 protein in a blood sample or in a sample of leukocytes extracted from said blood sample in said value is measured by cytofluorimetry using a monoclonal antibody specifically binding Plin2 and not binding other proteins labeled with a suitable fluorochrome,

c. comparing the value obtained in point a. with cutoff values of said concentration expressed in terms of mean fluorescence intensity (MFI) equal to 1.0 MFI; 1.4 MFI and 2.7 MFI

d. diagnosing NAS equal to 1 when the value obtained in a. is greater than or equal to the cutoff value of 1.0 MFI and lower than or equal to the cutoff value of 1.4 MFI; diagnosing NAS equal to 2 when the value obtained in a. is greater than the cutoff value of 1.4 MFI, or diagnosing NAS equal to 3 when the value obtained in a. is greater than the cutoff value of 2.7 MFI;

a method for the diagnosis of the severity grade of NASH, comprising the steps of

a. measuring the concentration value of the Plin2 protein in a blood sample or in a sample of leukocytes extracted from said blood sample in said value is measured by cytofluorimetry using a monoclonal antibody specifically binding Plin2 and not binding other proteins labeled with a suitable fluorochrome,

c. comparing the value obtained in point a. with cutoff values of said concentration expressed in terms of mean fluorescence intensity (MFI) equal to 2.7 MFI, 4 MFI and 6.3 MFI,

d. diagnosing NASH in mild form when the value obtained in a. is greater than the cutoff value of 2.7 MFI and lower than or equal to the cutoff value of 4 MFI, NASH in moderate form when the value obtained in a. is greater than the cutoff value of 4 MFI and lower than or equal to the value of 6.3 MFI, and NASH in severe form when the value obtained in a. is greater than 6.3 MFI;

said methods further comprising steps of measuring the protein concentration value of Pnpla3 and Rab14 proteins in a blood sample or in a sample of leukocytes extracted from said blood sample in point a. for which NASH was diagnosed, of comparison with respect to the concentration values thereof in blood samples coming from healthy patients and, optionally, from patients for whom the grade of hepatic fibrosis has been histologically defined, for the diagnosis of hepatic fibrosis; a method for monitoring the effectiveness of a therapeutic treatment of NAFLD or NASH on a patient, comprising the steps of

a. measuring the concentration value of the Plin2 protein in a blood sample or in a sample of leukocytes extracted from said blood sample of an individual suffering from NAFLD or NASH at an instant of time t0 from which said monitoring is carried out

b. measuring the concentration value of the Plin2 protein in a blood sample or in a sample of leukocytes extracted from said blood sample of an individual suffering from NAFLD or NASH at one or more time instants tn, wherein n is an integer greater than 0, and in which each tn corresponds to instants of time following to, wherein

a decrease in the concentration of the Plin2 protein in one or more of said time instants tn is indicative of an effectiveness of said therapeutic treatment;

a method for monitoring the progression of NAFLD or NASH on a patient, comprising the steps of

a. measuring the concentration value of the Plin2 protein in a blood sample or in a sample of leukocytes extracted from said blood sample of an individual suffering from NAFLD or NASH at an instant of time t0 from which said monitoring is carried out

b. measuring the concentration value of the Plin2 protein in a blood sample or in a sample of leukocytes extracted from said blood sample of an individual suffering from NAFLD or NASH at one or more time instants tn, wherein n is an integer greater than 0, and in which each tn corresponds to instants of time following t0, wherein

a decrease in the concentration of the Plin2 protein in one or more of said time instants tn is indicative of an improvement of the NAFLD or NASH, whereas an increase in the concentration of the Plin2 protein in one or more of said time instants tn is indicative of the deterioration of the NAFLD or NASH;

computer programs apt to implement said methods;

and a device for automatic measurement of the Plin2 protein concentration value in a blood sample comprising, in general:
isolation means of PBMCs from a blood sample;
measurement means of the concentration of the Plin2 protein in the cytoplasm of said PBMCs, and
a control unit connected to said isolation means and measurement means, programmed in such a way as to:
control and synchronize their actuation according to an automatic mode, according to predetermined operating parameters, and
automatically comparing the datum of the Plin2 protein concentration in the cytoplasm of said PBMCs with a reference datum of Plin2 protein concentration.

### Glossary

The term leukocytes for the purposes of the present invention has the meaning commonly known in the literature and comprises different cell types: granulocytes (or polymorphonuclears) which are subdivided into neutrophils, eosinophils or acidophils, basophils; lymphocytes; monocytes.

For the purposes of the present invention, the term Plin2 denotes the human protein Adipose differentiation-related protein, also known as ADFP, ADRP or perilipin 2 encoded by human gene ADFP located in 9p22.1.

### NAFLD Non-Alcoholic Hepatic Steatosis

### NASH Non-Alcoholic Steatohepatitis

Pnpla3 refers to human Pnpla3 protein, also known as adiponutrin, ADPN, Acylglycerol O-acyltransferase, C22orf20, IPLA2epsilon, DJ796I17.1, IPLA(2)epsilon, IPLA2-epsilon

Rab14 refers to human Rab14 protein, also known as Ras-related protein Rab-14.

### DETAILED DESCRIPTION OF THE FIGURES

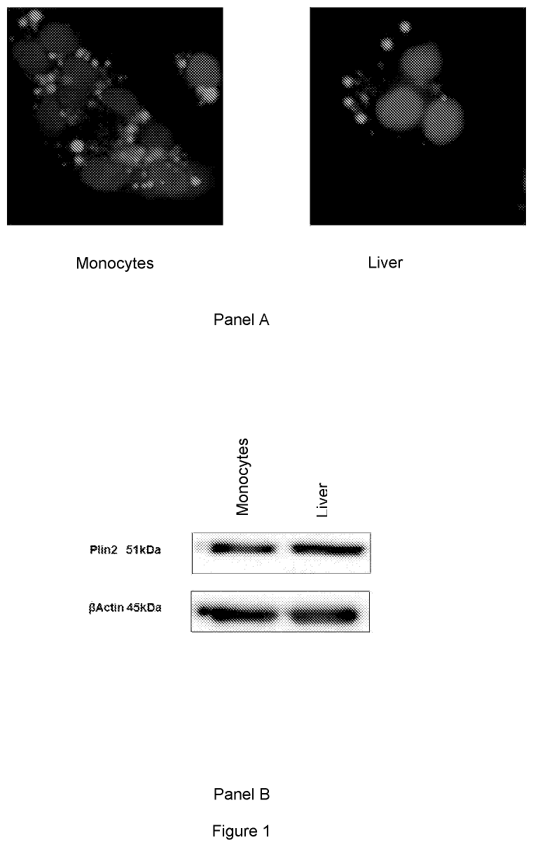

FIG. 1: Panel A: Lipid droplets (light-grey dots) in monocytes and hepatocytes: cell nuclei, larger and less intensely stained, are visible. Panel B shows a Western blot of Plin2 in monocytes and liver.

Beta actin shows how the protein amount detected in monocytes be similar to the protein amount detected in hepatocytes.

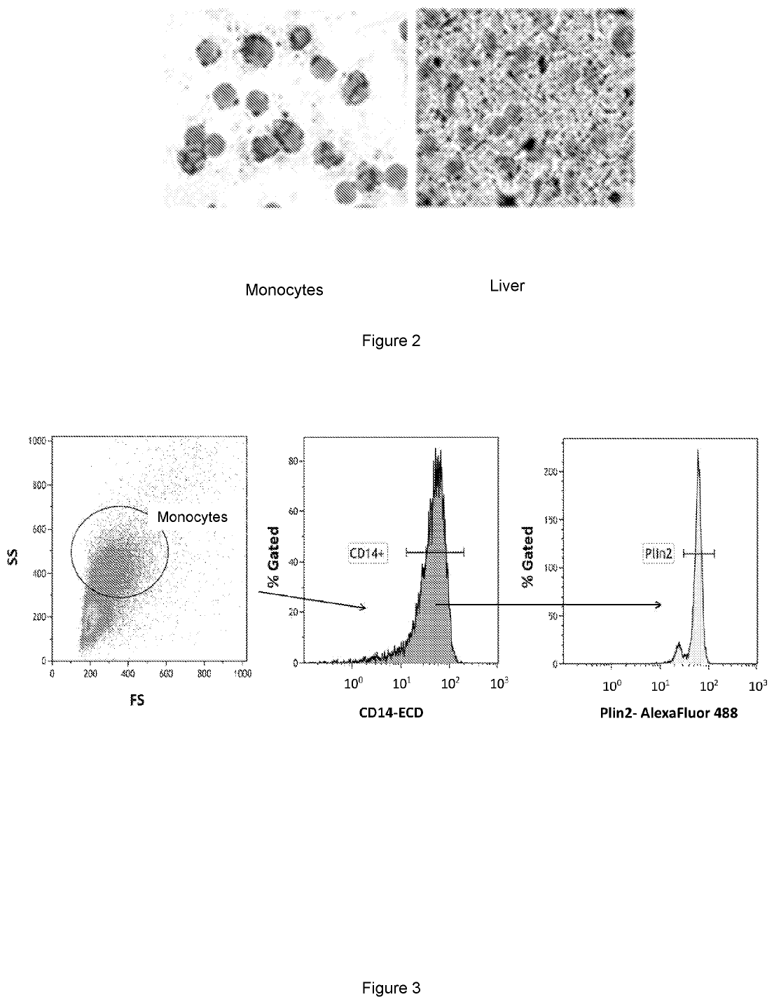

FIG. 2 reports ORO staining in a liver section and in monocytes of a same subject.

FIG. 3 reports a Bland Altman graph of differences (Y-axis) and means (X-axis) of Plin2 measurements by Western blot in monocytes and liver. The two dotted lines show congruence limits.

The difference between the values obtained with the two measurements is reported on the Y-axis, whereas the mean is on the Y-axis. Dotted lines report 95% of congruence bounds (limits) between the two measurements. The graph demonstrates that all points are inside the congruence limits, which demonstrates the direct correlation between Plin2 expression in liver and in peripheral blood cells.

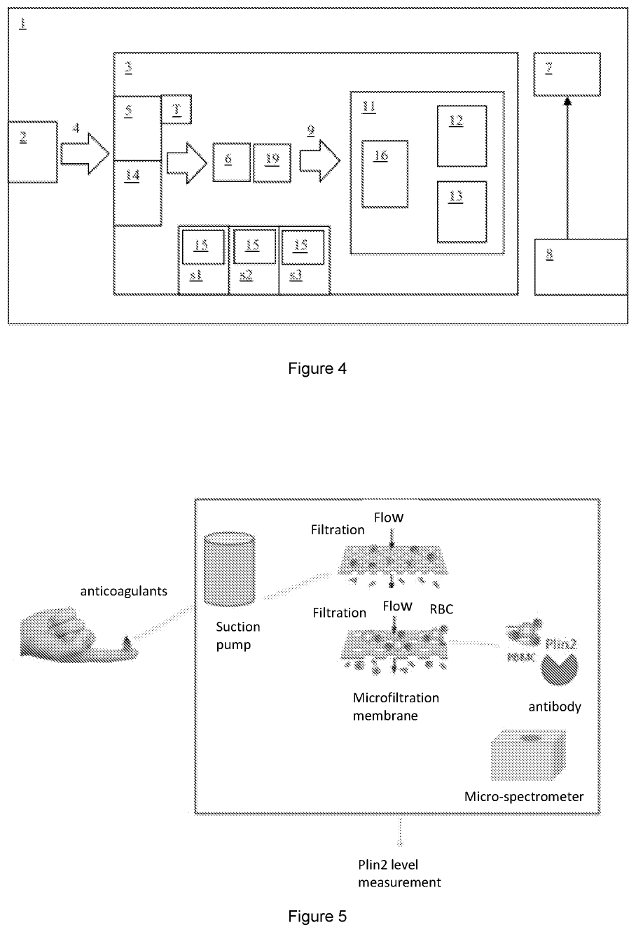

FIG. 4: Exemplary block diagram of a preferred embodiment of the device according to the present invention.

FIG. 5: exemplary diagram of use of a further preferred embodiment of the device according to the present invention.

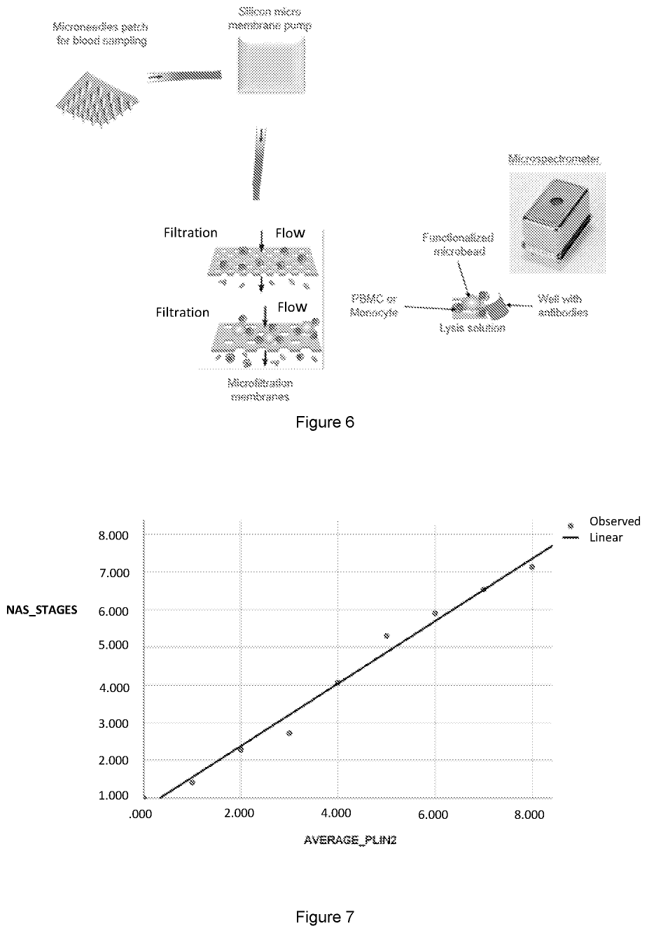

FIG. 6: exemplary diagram of use of a further preferred embodiment of the device according to the present invention

FIG. 7: correlation between mean Plin2 (MFI) levels and NAS stages, the independent variable, on the X-axis, is Plin2 moderated value, whereas the dependent one is the NAS stages.

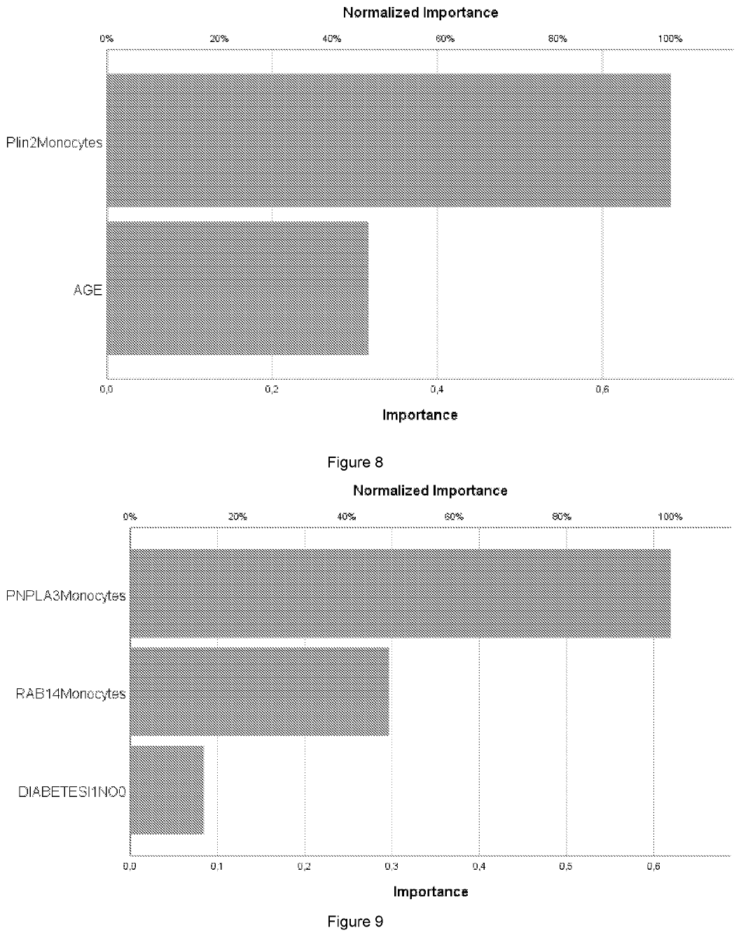

FIG. 8: Plin2 NASH normalized importance

FIG. 9: Pnpla3 and Rab14 Fibrosis normalized importance

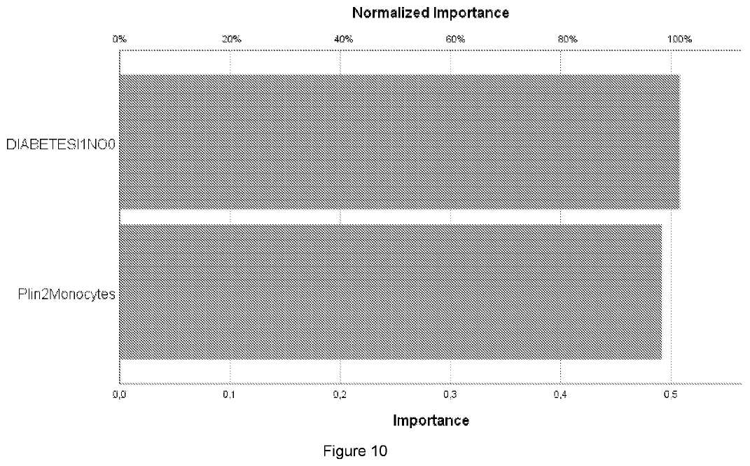

FIG. 10: Plin2 Fibrosis normalized importance

### DETAILED DESCRIPTION

The Authors of the present invention have surprisingly discovered that Plin2 protein is a NAFLD and NASH marker, that the protein concentration in blood cells (leukocytes) correlates with the expression of said protein in liver cells, and that its concentration is proportional to NAS score and also to the severity grade of the NASH disease. The Authors of the invention have therefore discovered that it is possible to use the measurement of the concentration of the Plin2 protein to perform a diagnosis of NAFLD or NASH, to diagnose the severity grade thereof as mild, moderate or severe, as defined in the literature, or to monitor the effectiveness of a therapeutic treatment of NAFLD or NASH, or also to monitor the course over time of NAFLD or NASH, by analysis of blood samples with no need to resort to liver biopsy.

Hence, the invention provides a method for the diagnosis of non-alcoholic hepatic steatosis (NAFLD) and/or or of non-alcoholic steatohepatitis (NASH) comprising the steps of

a. measuring the concentration value of the Plin2 protein in a blood sample or in a sample of leukocytes extracted from said blood sample of an individual

b. comparing the value obtained in point a. with the concentration value of the Plin2 protein in a blood sample or in a sample of leukocytes extracted from said blood sample of a healthy individual,

c. diagnosing NAFLD and/or NASH when the value measured in a. is greater than the value measured in b.

In one embodiment, in step a. a blood sample is used, that may be treated in order to isolate leukocytes therefrom by techniques known to a person skilled in the art.

The method can therefore comprise a step, preceding the measurement of the concentration of the Plin2 protein in the sample, wherein the blood sample is subjected to a treatment to isolate the leukocytes contained therein. In one embodiment, the method can comprise a step in which polymorphonuclear cells (polymorphonuclears) and/or monocytes are isolated from the blood sample to be analyzed or from the leukocytes isolated therefrom.

Any protocol known in the literature of current use for leukocyte isolation from a blood sample can be used.

Merely by way of example, polymorphonuclear cells can be isolated from a blood sample as follows: blood is collected into a test tube containing EDTA (final concentration 4 mM) to prevent coagulation thereof. Then, one or more discontinuous Percoll gradients (consisting of colloidal silica particles having a 15-30 nm diameter (23% w/w in water) that have been coated with polyvinylpyrrolidone (PVP)) are set up, placing 15 ml of 62% Percoll in a test tube and gently stratifying 15 ml of 75% Percoll therebelow, with the aid of a syringe provided with a thin catheter, avoiding the mixing of the two suspensions.

Then, 8-10 ml of blood are stratified on the above-described gradient; test tubes are centrifuged at 20° C. for 25 min overall, of which 10 min at 200 rpm and, next, 15 min at 400 rpm. Thus, the separation of the figurative elements of blood based on density and dimensions: erythrocytes and most of eosinophil granulocytes settle on the bottom of the test tube; polymorphonuclears settle at the interface between the two Percoll suspensions; lymphomonocytes localize between 62% Percoll and plasma.

After having discarded plasma and lymphomonocytes, the polymorphonuclears-containing band is collected with a glass Pasteur pipette, gathered in a test tube and subjected to washing in HEPES/BSA by centrifuging at 250 rpm, for 7 min, at 20° C. To discard possibly present erythrocytes, the bottom layer thus obtained is subjected to a rapid lysis treatment, resuspending it into 3 parts of hypotonic solution. After about 15 s of stirring, the medium isotonicity is restored by adding 7 parts of isotonic solution.

After a further washing at 250 rpm for 7 min, at 20° C., the cell pellet is resuspended and maintained into a known HEPES/BSA volume until use. Neutrophils concentration is determined by using an electronic particle counter, or under optical microscope using a hemocytometer.

Merely by way of example, various monocyte types can be isolated from the blood sample by the following techniques: via use of microbiologically expressed CD-specific recombinant Fab fragments of monoclonal antibodies against surface cell markers. Also, commercial kits are available for this purpose, such as, e.g., IBA GmbH™ CD14 Isolation Kit for FABian™.

The measurement of the Plin2 protein concentration can be performed according to any method available to a technician in the field, merely by way of example, in the methods described herein the measurement can be performed by Western blot, or Cytofluorimetry, or ELISA, or immunofluorescence or quantitative PCR, Enzyme-Linked Immunospot Assay) or localized surface plasmon resonance fiber tip probe system, or by using a device prepared for said measurement described hereinafter. All of the above-indicated methods are known to a technician in the field who, knowing on which sample they have to be performed and which protein should be quantitated, could select the protocol he/she deems most appropriate and use the most suitable reagents on the market. All reagents needed to perform in-sample Plin2 detection and quantitation according to the present description are known and available in the market, therefore requiring no specific contrivances by the technician in the field once the method of the invention is known.

By way of example, the above-listed techniques are summarized, and the steps normally used by the technician in the field for each of them are indicated.

Western Blotting enables to monitor protein expression in a cell, and therefore to determine the presence, the amount and the molecular weight of a specific antigen through three processes:

1) Protein extraction and dosage;
2) Protein separation by Sodium Dodecyl Sulphate—PolyAcrylamide Gel Electrophoresis (SD S-PAGE);
3) Western Blotting: transfer of proteins separated from the gel onto a nitrocellulose analysis (blot); exposure of the membrane to the antibody directed against the protein of interest (primary antibody); exposure of the membrane to an antibody directed against the antibody the species uses as primary antibody (secondary antibody), and membrane development with the chemiluminescence method (ECL).
4) Image densitometric analysis.

Cytofluorimetry is a laboratory technology allowing to detect, identify and count specific cells or proteins contained therein.

Cytofluorimetry techniques envisage various stages:

### Suspension of a Cell Sample in a Fluid

Before the test, and on the basis of the cells to be analyzed, the sample is treated with specific dyes capable of discriminating cell subtypes. This dye, fluorochrome, is bound to monoclonal antibodies directed to particular cell districts or marker antigens.

The sample containing labeled cells is introduced into the instrument referred to as cytofluorimeter.

In the instrument, the cell-containing fluid is channeled into the flow chamber and then through a very narrow hole, so as to create a flow inside which the cells are organized in a row, one after the other. The cell flow is placed in front of a detector, which thereby analyzes each cell present inside the flow at a very high rate (hundreds to thousands of cells per second).

The cytofluorimeter contains one or more lasers and several detectors able to identify some features, unique for each cell. Each laser impinges on cells present in the flow, generating for each of them a characteristic scatter, depending on the features thereof. The features can be physical (cell size and complexity) or can depend on the signal generated by the laser-intercepted dye (fluorochrome). The combination of this information generates a characteristic profile for each cell present inside the sample.

The signal detected by the detectors (or detector) is amplified (by photomultipliers) and sent to the computer. Here, it is converted into digital format and displayed on the computer or printed.

Data are provided in the form of graph.

ELISA (Enzyme-Linked Immunosorbent Assay) is a highly used technique, based on the chemical conjugation of enzymes with antibodies or antigens. The activity of these enzymes is easily monitorable and allows to accurately quantitate conjugate complex concentration. Depending on the specific method used, ELISA can be used for dosing antigens or antibodies.

Antigen Recognized by the Specific Antibody (Immuno)

Analyte (Antigen or Antibody) Adsorbed on System (Sorbent) Surface

Antigen/antibody recognized by a (second) enzyme-linked antibody, able to give a reaction whose product is stained.

Quantitative PCR or real-time PCR is a technology used to quantitate nucleic acids, in this case mRNA encoding the Plin2 protein, through the measurement of fluorescence emitted by a fluorophore. This technique associates amplification and quantitation within a single reaction. In a real-time PCR reaction, fluorescence increases in proportion to PCR products' accumulation. The technician in the field will have no problem at all in designing probes suitable for carrying out the RT-PCR, as the gene and the DNA encoding for the Plin2 protein are known in the literature. Enzyme-linked immunospot (ELISPOT) analysis in a PBMCs sample is based on cell incubation for a definite period of time, in a 96-well plate previously functionalized (coating) by adsorption of a monoclonal antibody with high affinity for the investigated cytokine that is produced during the incubation. The combination of an anti-reference protein biotinylated antibody and of an anti-biotin enzyme-conjugated secondary antibody, in thy: presence of chromogenic substrates for the enzyme, enables detection of protein production in defined areas due to reaction product precipitation by formation of spots (dye accumulations). Said technique can be applied to PBMCs content after cell lysis.

Surface plasmons were used to improve surface sensitivity of various spectroscopic measurements, including fluorescence, Raman scattering and second harmonic generation. However, in their simplest form, SPR reflectance can be used to detect molecular absorption in proteins, etc. Technically, the minimum reflection angle (Absorption maximum) is measured.

Any analytical technique known in the literature, suitable to quantitate Plin2 concentration in the blood or cell sample as above-defined can be used in any method of the invention.

Furthermore, the device according to the present invention can be designed to perform Plin2 protein quantitation in the sample of interest by one or more of the above-described detection techniques.

In a particular embodiment, the measurement can be performed by cytofluorimetry, using as protein marker an antibody (or a derivative or fragment thereof as above-defined) specific for the Plin2 protein, i.e. binding only Plin2 protein, labeled with a suitable fluorochrome.

In one embodiment, the fluorochrome may be any one fluorochrome detectable by marketed cytofluorimetry apparatuses, like, e.g. a fluorochrome having the following features:

| | Absorption | Emission | MM | ε | Quantum |
|---|---|---|---|---|---|
| Color | (nm) | (nm) | (g/mol) | (cm⁻¹M⁻¹) | Yield |
| Cyan-green | 495 | 519 | 643 | 73.000 | 0.92 |

In one embodiment, Alexa Fluor 488 fluorochrome (Invitrogen) can be used, commonly used as an alternative to FITC or to Cyt, having the following technical features.

The analysis of fluorescence intensity and the mean fluorescence intensity (MFI) value can be computed by suitable software available to the public and assessed by the use of cytofluorimeters available on the market.

Therefore, according to one embodiment, the fluorochrome used can be Alexa Fluor 488, with the above-defined technical features, the cytofluorimeter FC 500 (Beckman Coulter, Brea, Calif.) and the data can be analyzed with Kaluza software (Beckman Coulter, Brea, Calif.) with the technical features of said products at the time of filing of the present application.

The technician in the field would anyhow know how to adapt cutoffs expressed in terms of MFI provided herein, computed by using Alexa Fluor 488 fluorochrome or a fluorochrome with the features reported in the above Table, using the cytofluorimeter FC 500 (Beckman Coulter, Brea, Calif.) or a cytofluorimeter with similar technical features and the Kaluza software (Beckman Coulter, Brea, Calif.) at cutoffs expressed in terms of MFI computed using other fluorochromes and other devices and software, by a comparative assay.

The MFI is meant as a simple arithmetic mean.

According to the present invention, the cytofluorimeter can preferably have the technical features of the above-indicated cytofluorimeter, as available to the public at the time of filing of the present application.

All embodiments for the above-described steps are applicable to any of the methods provided in the present description.

In one embodiment, the invention relates to a method for the diagnosis of NAFLD or NASH, comprising the steps of

a. measuring the concentration value of the Plin2 protein in a blood sample or in a sample of leukocytes extracted from said blood sample

b. measuring the concentration value of the Plin2 protein in a population of blood samples coming from patients suffering from NAFLD and of healthy patients and identifying a cutoff value, C0, of said concentration between patients suffering from NAFLD and healthy patients, and measuring the concentration value of the Plin2 protein in a population of blood samples coming from patients suffering from NASH and of healthy patients and identifying a cutoff value, C1, of said concentration between patients suffering from NASH and healthy patients,

c. comparing the value obtained in point a. with the cutoff values obtained in point b

d. diagnosing NAFLD when the value obtained in a. is greater than or equal to said value C0 and lower than said value C1, or diagnosing NASH when the value obtained in a. is greater than or equal to said value C1.

The invention also relates to a method for the diagnosis of the NAS score of patients suffering from NAFLD or NASH, comprising the steps of

a. measuring the concentration value of the Plin2 protein in a blood sample or in a sample of leukocytes extracted from said blood sample

b. measuring the concentration value of the Plin2 protein in a population of blood samples coming from patients suffering from NAFLD or NASH and of healthy patients wherein, in said population comprising samples coming from patients suffering from NAFLD, the NAS score value was histologically defined in said patients as NAS 1, NAS 2 and NAS 3, on the basis of the percentage of steatosis in liver cells, the occurrence of ballooned hepatocytes, the presence of lobular inflammation and the presence of portal inflammation and identifying:

a cutoff value, C2, of said concentration between healthy patients and patients suffering from NAFLD with NAS score 1;

a cutoff value, C3, of said concentration between patients suffering from NAFLD with NAS 1 and patients suffering from NAFLD with NAS score 2; and

a cutoff value, C4, of said concentration between patients suffering from NAFLD with NAS 2 and patients suffering from NASH with NAS score 3,

c. comparing the value obtained in point a. with the cutoff values obtained in point b

d. diagnosing the NAS score as

NAS score 1 when the value obtained in a. is greater than or equal to said cutoff value C2 and lower than or equal to said cutoff value C3,

NAS score 2 when the value obtained in a. is greater than said value C2 and lower than or equal to said cutoff value C4,

NAS score 3 when the value obtained in a. is greater than said value C4.

The NAS score according to the present invention is as defined in the literature and is normally assigned according to the following parameters:

| NAS | | Lobular | Ballooning |
|---|---|---|---|
| SCORE | Steatosis | inflammation | degeneration |
| 0 | <5% | Absent | Absent |
| 1 | 5-33% | <2foci/20x field | Rare |
| 2 | >33-66% | 2-4foci/20x field | Frequent |
| 3 | >66% | foci/20x field | |

The invention also relates to a method for the diagnosis of the severity grade of NASH, comprising the steps of

a. measuring the concentration value of the Plin2 protein in a blood sample or in a sample of leukocytes extracted from said blood sample

b. measuring the concentration value of the Plin2 protein in a population of blood samples coming from patients suffering from NASH and of healthy patients wherein, in said population comprising samples coming from patients suffering from NASH, the severity grade of the pathology was histologically defined in said patients as mild, moderate or severe on the basis of the percentage of steatosis in liver cells, the occurrence of ballooned hepatocytes, the presence of lobular inflammation and the presence of portal inflammation and identifying

a cutoff value, C5, of said concentration between healthy patients and patients suffering from mild NASH;

a cutoff value, C6, of said concentration between patients suffering from mild NASH and patients suffering from moderate NASH; and

a cutoff value, C7, of said concentration between patients suffering from moderate NASH and patients suffering from severe NASH,

c. comparing the value obtained in point a. with the cutoff values obtained in point b,

d. diagnosing the severity grade of NASH as

mild when the value obtained in a. is greater than said value C5 and lower than said value C6,

moderate, when the value obtained in a. is greater than said value C6 and lower than said value C7, and

severe, when the value obtained in a. is greater than said value C7.

Hence, the cutoff values of Plin2 concentration according to the present description are:

cutoff between patients suffering from NAFLD and healthy patients C0

cutoff between patients suffering from NASH and healthy patients C1

cutoff between healthy patients and patients suffering from NAFLD with NAS 1 C2

cutoff between patients suffering from NAFLD with NAS 1 and patients suffering from NAFLD with NAS 2 C3

cutoff between patients suffering from NAFLD with NAS 2 and patients suffering from NASH with NAS 3 C4

cutoff between healthy patients and patients suffering from mild NASH C5

cutoff between patients suffering from mild NASH and patients suffering from moderate NASH C6

cutoff between patients suffering from moderate NASH and patients suffering from severe NASH C7.

The definition of the forms of severity of the disease according to the invention is that commonly used in the literature on the basis of histological analyses according to the NAFLD Activity Score (NAS) (Kleiner D E, Brunt E M, Van Natta M, Behling C, Contos M J, Cummings O W, et al. Design and validation of a histological scoring system for nonalcoholic fatty liver disease. HEPATOLOGY 2005; 41:1313-1321.) It is a histological score, widely used and confirmed by hepatology societies worldwide, based on the presence in liver biopsies of steatosis, lobular and portal inflammation and ballooned hepatocyte degeneration (“ballooning”); each component can be of grade 1 (mild), 2 (moderate) and 3 (severe).

Grading of NASH EM Brunt Sem Liver Dis 2001; 21:3-16

| | | Hepatocellular | Lobular | Portal |
|---|---|---|---|---|
| GRADE | Steatosis | ballooning | inflammation | inflammation |
| Mild | ≤66%; | Zone 3; | Scattered | None or mild |
| | mostly | Occasional | polys and | |
| | macrovesicular | cells | mononuclear | |
| | | | cells | |
| Moderate | ≤66%; | Zone 3; | Polys with | Mild to |
| | usually | obvious | ballooned | moderate |
| | mixed | | cells & | |
| | | | areas of | |
| | | | pericellular | |
| | | | fibrosis | |
| Severe | ≥66%; | Predominantly | Polys with | Mild to |
| | usually | Zone 3; | ballooned | moderate |
| | mixed | marked | cells & | |
| | | | areas of | |
| | | | pericellular | |
| | | | fibrosis | |

In fact, the Authors of the present invention have surprisingly discovered that the concentration value of the Plin2 protein in analyzed blood samples from patients whose NASH severity grade is known correlates with said grade, and that the increase of Plin2 protein concentration is directly proportional to the severity of the pathology.

In a preferred embodiment, the above-described methods are carried out by using the cytofluorimetric technique to assess the Plin2 protein expression, using a monoclonal antibody specifically binding Plin2 and not binding other proteins labeled with a suitable fluorochrome, and said cutoff is expressed in terms of mean fluorescence intensity (MFI).

In a more preferred embodiment, it is by using the above-described fluorochromes, software and devices, or also the above-described fluorochromes and the software and the devices subject-matter of the present invention as defined hereinafter and in the claims. According to the literature and according to the invention, NASH necroinflammatory grades are classified as grade 1 (mild), grade 2 (moderate) and grade 3 (severe) on the basis of the grade of hepatocellular steatosis, ballooning and disorder, and (intralobular and portal) inflammation (Table above).

The Authors of the present invention have demonstrated (see Examples section) that specific cutoffs (expressed herein in terms of MFI with the fluorochrome, the software and the device defined hereinafter) applicable to the above-described methods can be computed.

Hence, object of the invention are also:

a method for the diagnosis of NAFLD or NASH, comprising the steps of

a. measuring the concentration value of the Plin2 protein in a blood sample or in a sample of leukocytes extracted from said blood sample, wherein said value is measured by cytofluorimetry using a monoclonal antibody specifically binding Plin2 and not binding other proteins labeled with a suitable fluorochrome

b. comparing the value obtained in point a. with a cutoff value of said concentration expressed in terms of mean fluorescence intensity (MFI) equal to 2.7 MFI and with a cutoff value of said concentration equal to 1.0 MFI

d. diagnosing NAFLD when the value obtained in a. is greater than or equal to 1.0 MFI and lower than or equal to 2.7 MFI, or diagnosing NASH when the value obtained in a. is greater than 2.7 MFI.

A method for the diagnosis of the NAS score of patients suffering from NAFLD or NASH, comprising the steps of

a. measuring the concentration value of the Plin2 protein in a blood sample or in a sample of leukocytes extracted from said blood sample in said value is measured by cytofluorimetry using a monoclonal antibody specifically binding Plin2 and not binding other proteins labeled with a suitable fluorochrome,

c. comparing the value obtained in point a. with cutoff values of said concentration expressed in terms of mean fluorescence intensity (MFI) equal to 1.0 MFI; 1.4 MFI and 2.7 MFI

d. diagnosing NAS score 1 when the value obtained in a. is greater than or equal to the cutoff value of 1.0 MFI and lower than or equal to the cutoff value of 1.4 MFI; diagnosing NAS score 2 when the value obtained in a. is greater than the cutoff value of 1.4 MFI, or diagnosing NAS score 3 when the value obtained in a. is greater than the cutoff value of 2.7 MFI; and

a method for the diagnosis of the severity grade of NASH, comprising the steps of

a. measuring the concentration value of the Plin2 protein in a blood sample or in a sample of leukocytes extracted from said blood sample in said value is measured by cytofluorimetry using a monoclonal antibody specifically binding Plin2 and not binding other proteins labeled with a suitable fluorochrome,

c. comparing the value obtained in point a. with cutoff values of said concentration expressed in terms of mean fluorescence intensity (MFI) equal to 2.7 MFI, 4 MFI and 6.3 MFI

d. diagnosing NASH in mild form when the value obtained in a. is greater than the cutoff value of 2.7 MFI and lower than or equal to the cutoff value of 4 MFI, NASH in moderate form when the value obtained in a. is greater than the cutoff value of 4 MFI and lower than or equal to the value of 6.3 MFI, and NASH in severe form when the value obtained in a. is greater than 6.3 MFI.

Such Methods are One Embodiment

using the Alexa Fluor 488 fluorochrome, or a fluorochrome with the features reported in the above table, using the FC 500 cytofluorimeter (Beckman Coulter, Brea, Calif.) and Kaluza software (Beckman Coulter, Brea, Calif.). Likewise, different cutoffs, C0-C7, can be defined by using different detection and quantitation systems of the Plin2 protein.

A indicated above, the Authors have also surprisingly discovered that the assessment of the concentration of Pnpla3 and Rab14 proteins in a blood sample or in a sample of leukocytes extracted from said blood sample in point a. for which NASH was diagnosed enables to define whether hepatic fibrosis is present or absent, and the severity grade thereof.

In the present invention, in order to define hepatic fibrosis the same definition used in the literature (Kleiner D E, Brunt E M, Van Natta M, Behling C, Contos M J, Cummings O W, et al. Design and validation of a histological scoring system for nonalcoholic fatty liver disease. HEPATOLOGY 2005; 41:1313-1321) is used, based on a score system for staging on the basis of fibrosis position and extension: stage 1, zone 3 perisinusoidal fibrosis; stage 2, portal fibrosis with the abovementioned stage 1; stage 3, bridging fibrosis besides stage 2; and stage 4, cirrhosis. NASH Clinical Research Network (NASH CRN) has subsequently subdivided stage 1 into 3 categories: stage 1A, mild perisinusoidal fibrosis in zone 3; stage 1B, moderate perisinusoidal fibrosis in zone 3; and stage 1C, portal/periportal fibrosis only. Stage 1C fibrosis is observed occasionally in children or in severely obese patients.

| Histological alteration | Fibrosis stage (SAF F) |
|---|---|
| Absence of fibrosis | 0 |
| Perisinusoidal or periportal fibrosis | 1 |
| Perisinusoidal or periportal fibrosis | 2 |
| Bridging fibrosis | 3 |
| Cirrhosis | 4 |

Hence, the invention also provides further steps to be applied to the above-reported methods, wherein, once NASH is diagnosed from a patient's blood sample, it is possible also to diagnose, from the same sample or from a further blood sample of said patient, the presence or absence of fibrosis and the pathological stage thereof.

The steps of fibrosis stage diagnosis can also be made on blood samples, as defined in the present description and in the claims, of patients suffering from NASH, in which the diagnosis of NASH has been performed with a method different from that described and claimed in the present invention.

Therefore, step e. can be replaced, in any of the embodiments described herein, by step e′. measuring the concentration value of Pnpla3 and Rab14 proteins in a blood sample or in a sample of leukocytes extracted from a blood sample of a patient diagnosed with NASH and step g. can therefore be replaced by step g′. comparing the value obtained in point e′. with the cutoff value obtained in point f.

Therefore, object of the invention is any one of the methods for the diagnosis of sole NASH and/or of the seventy thereof, further comprising the steps of

e. measuring the concentration value of Pnpla3 and Rab14 proteins in a blood sample or in a sample of leukocytes extracted from said blood sample in point a. for which NASH was diagnosed

f. measuring the concentration value of Pnpla3 and Rab14 proteins in a population of blood samples coming from patients suffering from hepatic fibrosis and healthy patients and identifying a cutoff value, C8, of said concentration between patients suffering from hepatic fibrosis and healthy patients,

g. comparing the value obtained in point e. with the cutoff value obtained in point f

h. diagnosing hepatic fibrosis when the value obtained in point e. is greater than said value C8, or a method for the diagnosis of hepatic fibrosis comprising steps e′, f, g′ and h. Object of the invention is also any one of the methods for the diagnosis of sole NASH and/or of the severity thereof, further comprising the steps

e. measuring the protein concentration value of Pnpla3 and Rab14 proteins in a blood sample or in a sample of leukocytes extracted from said blood sample in point a. for which NASH was diagnosed

f. measuring the concentration value of Pnpla3 and Rab14 proteins in a population of blood samples coming from healthy patients and from patients suffering from hepatic fibrosis, wherein the severity grade of said pathology was histologically defined as stage 1, stage 2 or stage 3 on the basis of the position and extension of the hepatic fibrosis, and identifying

a cutoff value, C9, of said concentration between healthy patients and patients suffering from stage 1 hepatic fibrosis;

a cutoff value, C10, of said concentration between patients suffering from stage 1 hepatic fibrosis and patients suffering from stage 2 hepatic fibrosis; and

a cutoff value, C11, of said concentration between patients suffering from stage 2 hepatic fibrosis and patients suffering from stage 3 hepatic fibrosis,

g. comparing the value obtained in point e. with the cutoff values obtained in point f

h. diagnosing the stage of hepatic fibrosis as

stage 1 when the value obtained in point e. is greater than or equal to said value C9 and lower than said value C10,

stage 2 when the value obtained in point e. is greater than or equal to said value C10 and lower than or equal to said value C11, and

stage 3 when the value obtained in point e. is greater than said value C11, or a method for the diagnosis of the stage of hepatic fibrosis comprising steps e′, f, g′ and h′, wherein all references to point e. of step h. above are changed into reference to point e′, with the necessary modifications.

Object of the invention are also a method for the diagnosis of hepatic fibrosis and a method for the diagnosis of the stage of hepatic fibrosis on blood samples or on lymphocytes from patients diagnosed with NASH comprising the above-described steps, wherein steps e and g are replaced by steps e′ and g′.

To sum up, the cutoff values of Pnpla3 and Rab14 concentration as defined herein are:

cutoff between patients suffering from hepatic fibrosis and healthy patients C8

cutoff between healthy patients and patients suffering from stage 1 hepatic fibrosis C9

cutoff between patients suffering from stage 1 hepatic fibrosis and stage 2 hepatic fibrosis C10

cutoff between patients suffering from stage 1 hepatic fibrosis and stage 3 hepatic fibrosis C11

The concentration value of the abovementioned proteins can be measured by Western blot, or Cytofluorimetry, or ELISA, or quantitative PCR.

In a particular embodiment, it can be measured by cytofluorimetry using a monoclonal antibody specifically binding Pnpla3 and not binding other proteins, and a monoclonal antibody specifically binding Rab14 and not binding other proteins, labeled with a suitable fluorochrome, and said cutoff is expressed in terms of mean fluorescence intensity (MFI).

As mentioned above, the concentration value of Pnpla3 and Rab14 proteins can be computed by using all of the embodiments and devices described for the computing of the value of Plin2 concentration, such as, e.g., as fluorochrome having the following features:

| | Absorption | Emission | MM | ε | Quantum |
|---|---|---|---|---|---|
| Color | (nm) | (nm) | (g/mol) | (cm⁻¹M⁻¹) | Yield |
| Cyan-green | 495 | 519 | 643 | 73.000 | 0.92 |

Therefore, according to one embodiment, the fluorochrome used can be Alexa Fluor 488, with the above-defined technical features, the cytofluorimeter FC 500 (Beckman Coulter, Brea, Calif.) and the data can be analyzed with Kaluza software (Beckman Coulter, Brea, Calif.) with the technical features of said products as available to the public at the time of filing of the present application.

The Authors of the present invention have demonstrated (see Examples section) that specific cutoffs (expressed herein in terms of MFI with the fluorochrome, the software and the device defined hereinafter) applicable to the above-described methods can be computed.

Therefore, objects of the invention are also any one of the methods for the diagnosis of sole NASH and/or of the severity thereof, further comprising the steps of

e. measuring the concentration value of Pnpla3 and Rab14 proteins in a blood sample or in a sample of leukocytes extracted from said blood sample in point a. for which NASH was diagnosed, wherein said value is measured by cytofluorimetry using a monoclonal antibody specifically binding PNPLA and not binding other proteins, and a monoclonal antibody specifically binding Rab14 and not binding other proteins, labeled with a suitable fluorochrome,

g. comparing the value obtained in point e. with a cutoff value, expressed in terms of mean fluorescence intensity (MFI) greater than or equal to 1.24 MFI

h. diagnosing hepatic fibrosis when the value obtained in point e. is greater than or equal to 1.24 MFI

and any one of the methods for the diagnosis of sole NASH and/or of the severity thereof, further comprising the steps of

e. measuring the concentration value of Pnpla3 and Rab14 proteins in a blood sample or in a sample of leukocytes extracted from said blood sample in point a. for which NASH was diagnosed, wherein said value is measured by cytofluorimetry using a monoclonal antibody specifically binding PNPLA and not binding other proteins, and a monoclonal antibody specifically binding Rab14 and not binding other proteins, labeled with a suitable fluorochrome, and said cutoff is expressed in terms of mean fluorescence intensity (MFI),

g. comparing the value obtained in point a. with cutoff values of said concentration expressed in terms of mean fluorescence intensity (MFI) equal to 1.24 MFI, 2.3 MFI and 3.10 MFI

h. diagnosing mild stage 1 hepatic fibrosis when the value obtained in point e. is greater than or equal to 1.24 MFI and lower than 2.4 MFI, stage 2 fibrosis when the value obtained in point e. is greater than or equal to 2.4 MFI and lower than 3.10 MFI, and stage 3 hepatic fibrosis when the value obtained in point e. is greater than or equal to 3.10 MFI.

In this case as well, object of the invention are also a method for the diagnosis of hepatic fibrosis and a method for the diagnosis of the stage of hepatic fibrosis on blood samples or on lymphocytes from patients diagnosed with NASH comprising the above-described steps, wherein steps e. and g. are replaced by steps e′ and g′ and h′, wherein all references to point e. of step h. above are changed into references to point e′, with the necessary modifications.

The above-reported values were defined by using Alexa Fluor 488 fluorochrome, or a fluorochrome with the features reported in the above table, using the Cytofluometer FC 500 (Beckman Coulter, Brea, Calif.) and Kaluza software (Beckman Coulter, Brea, Calif.). Clearly, other cutoff values can be assigned by using different detection systems.

The technician in the field would anyhow know how to adapt cutoffs expressed in terms of MFIs provided herein, computed by using the Alexa Fluor 488 fluorochrome or a fluorochrome with the features reported in the above table, using the Cytofluometer FC 500 (Beckman Coulter, Brea, Calif.) and Kaluza software (Beckman Coulter, Brea, Calif.) at cutoffs computed using other fluorochromes and other devices and software, by a comparative assay.

Furthermore, the invention provides a method for monitoring the effectiveness of a therapeutic treatment of NAFLD or of NASH on a patient, comprising the steps of

a. measuring the concentration value of the Plin2 protein in a blood sample or in a sample of leukocytes extracted from said blood sample of an individual suffering from NAFLD or NASH at an instant of time t0, from which said monitoring is carried out

b. measuring the concentration value of the Plin2 protein in a blood sample or in a sample of leukocytes extracted from said blood sample of an individual suffering from NAFLD or NASH at one or more time instants tn, wherein n is an integer greater than 0, and in which each tn corresponds to instants of time following t0, wherein

a decrease in the concentration of the Plin2 protein in one or more of said time instants tn is indicative of an effectiveness of said therapeutic treatment.

In the carrying out of the above-described monitoring method, time t0 is the time in which the monitoring is started, which can correspond at an instant of time preceding the beginning of the therapy, or can be a time instant from which the monitoring is started, even once therapy has begun.

The instants of time tn, are instants of time following (next to) each other with the increase of the value of n, and following instant t0.

Hence, the instant of time t0 is the instant at which the monitoring is started, and the concentration value of Plin2 in the blood sample is considered as the value from which the assessing of the therapy effectiveness begins. The measurement of the concentration is then repeated at times following t0 and following (next to) each other in progression from 1 onwards, said times tn, wherein n is an integer greater than 0, whereby the instant of time t1 precedes the instant of time t2 which precedes the instant of time t3, and so on.

A detectable and significant decrease of Plin2 concentration in the blood samples of the patient analyzed in the times following t0 with respect to the value measured in t0 is indicative of an effectiveness of the therapeutic treatment in improving NAFLD or NASH. The maintenance of a value constant over time is indicative of an effectiveness of the therapeutic treatment in containing NAFLD or NASH, an increase of the value over time with respect to that measured in t0 is indicative of an ineffectiveness of the therapeutic treatment.

In some cases, prior to performing a pharmacological therapy, a change in food habits and lifestyle is suggested to the patient. In these cases, it can be useful to monitor over time the progression of the disease, to see, e.g., if the change in food habits and lifestyle positively affect the disease.

Monitoring anyhow the course of the disease, regardless of assessing or not assessing a possible therapeutic effectiveness, can be of interest for the treating physician. Hence, object of the invention is also a method for monitoring the progression over time of NAFLD or of NASH in a patient, comprising the steps of

a. measuring the concentration value of the Plin2 protein in a blood sample or in a sample of leukocytes extracted from said blood sample of an individual suffering from NAFLD or NASH at an instant of time t0 from which said monitoring is carried out

b. measuring the concentration value of the Plin2 protein in a blood sample or in a sample of leukocytes extracted from said blood sample of an individual suffering from NAFLD or NASH at one or more time instants tn, wherein n is an integer greater than 0, and in which each tn corresponds to instants of time following t0, wherein

a decrease in the concentration of the Plin2 protein in one or more of said time instants tn is indicative of an improvement of the NAFLD or NASH, whereas an increase in the concentration of the Plin2 protein in one or more of said time instants tn is indicative of the deterioration of the NAFLD or NASH, whereas a constant maintenance of the value over time is indicative of a situation of disease stasis.

What described on the instants of time t0 and tn in the explanation of the method for the monitoring of the therapeutic treatment, applies to the method for monitoring in general.

All embodiments described for the diagnostic method also apply to the monitoring methods described hereinafter.

In general, though blood samples may be collected also from organs, etc., in the carrying out of the methods described herein the use of peripheral blood is preferred.

In particular, in the implementation of the methods described herein it is preferable to perform the measurement of the concentration of Plin2, Pnpla3, Rab14 on the leukocytes isolated from said peripheral blood, and more particularly on polymorphonuclear cells (polymorphonuclears) and/or monocytes.

The same methods for monitoring the effectiveness and the disease progression indicated above for NAFLD or NASH can be performed, with the necessary modifications, at the monitoring of the therapeutic effectiveness or of the progression of hepatic steatosis using as marker the Pnpla3 and Rab14 concentration values in blood samples of patients suffering from hepatic steatosis.

The present invention moreover provides a device for automatic measurement of the of the Plin2 protein concentration value and/or the Pnpla3 and/or Rab14 concentration values in a blood sample collected from an individual, comprising, in general:

isolation means of PBMCs from a blood sample;

measurement means of the concentration of the Plin2 and/or Pnpla3 and/or Rab14 protein in the cytoplasm of said PBMCs, and

means correlating the obtained measurement to a comparative value that can be a value obtained from healthy individuals and/or one or more values of the concentration of the Plin2 and/or Pnpla3 and/or Rab14 protein in the cytoplasm di PBMC from blood samples of the same individual, previously obtained.

As mentioned above, the device according to the present invention can be implemented so as to perform the measurement of the Plin2 and/or Pnpla3 and/or Rab14 protein in a blood sample taken from an individual by one or more of the above-described detection techniques.

Depending on the selected technique for PBMCs isolation and lysis, the abovementioned means will be implemented in accordance with various variants, some of which will be described hereinafter. For instance, in case a form of separation by components selectively binding PBMCs or monocytes is used, the device could comprise:
a collection chamber apt to receive the blood sample;
mixing means comprised in said collection chamber, configured to mix said blood sample with a component which is apt to bind to polymorphonuclears (PBMCs) present in said sample, and, where necessary, with an anticoagulating substance;
moving means configured to apply a mechanical vibration and/or rotation stress to the mixture obtained by said mixing means and to convey said mixture to filtering means;
said filtering means configured to retain said component, which is bound to said polymorphonuclears (PBMCs) and allow the rest of said mixture to be eluted;
isolation means of said polymorphonuclears (PBMCs) from the component to which they are bonded, configured to favor the separation between said polymorphonuclears (PBMCs) and said component;
treatment means of the polymorphonuclears (PBMCs) isolated by said isolation means, configured in such a way as to allow the binding of the Plin2 and/or Pnpla3 and/or Rab14 protein, when present, with a reagent specific for said protein, wherein said reagent is fluorescent at one or more predetermined wavelengths;
at least one radiation source, configured to emit radiations at a predetermined wavelength in such a manner that said reagent emits fluorescence, e.g. in the form of light waves;
means for acquiring images of the irradiated sample;
a control unit (7), connected to all said means and to said radiation source (12), programmed so as to control and synchronize their actuation according to an automatic mode, according to predetermined operating parameters, wherein said control unit is further configured to process said images so as to quantitate the Plin2 and/or Pnpla3 and/or Rab14 protein concentration present in said sample.

Said control unit will be further configured to compare said measured concentration with one or more reference data of the Plin2 and/or Pnpla3 and/or Rab14 protein concentration, obtained from measurements performed on blood samples of healthy subjects and/or on blood samples of the same individual collected at different instants of time.

In one embodiment, with reference to the Plin2 protein, for instance, said reference datum of Plin2 protein concentration is a datum associated with a healthy subject and wherein said control unit 7 is further programmed to detect a non-alcoholic hepatic steatosis (NAFLD) condition if the datum of the Plin2 protein concentration obtained from the sample analysis is greater than said reference datum of Plin2 protein concentration.

Analogously to what has been described above, the degree of concentration of Pnpla3 and Rab14 proteins can be used to detect a hepatic fibrosis condition and optionally assess the severity thereof. According to a further embodiment, said reference datum of Plin2 protein concentration is a datum associated with the same patient and detected in a time instant (t0) preceding a time instant (tn) in which said blood sample was detected, and wherein said control unit 7 is further programmed to detect the course of the disease over time.

For instance, if the datum of the Plin2 protein concentration obtained from the sample analysis is greater than the reference datum, an improvement of NAFLD is detected;

if the datum of the Plin2 protein concentration obtained from the sample analysis is lower than the reference datum, a deterioration of NAFLD is detected, whereas, if the datum of the Plin2 protein concentration obtained from the sample analysis is equal to the reference datum, a disease stasis is detected.

Preferably, the datum of the Plin2 protein concentration obtained from the sample analysis is considered to be equal to the reference datum when the difference between the two datums is of from about 0 to about 5%.

A first and a second preferred embodiment of the device are illustrated by way of example in FIGS. 4 and 5.

According to one embodiment, a blood sample, preferably already mixed with an anticoagulant, is introduced into a collection chamber 3 internal to the device. Preferably, the device 1 can comprise puncture means 2 apt to pierce the skin of an individual from which said blood sample is to be taken. For instance, the puncture means 2 can be suitable to pierce the patient's skin at a fingertip. Said puncture means comprises in particular at least a protruding element ending with a pointed end, e.g. a needle. The puncture means 2 is carried at an external surface of the device 1, and can be configured and articulated in a way such as to be able to assume a configuration of minimum encumbrance in a non-use condition, in which the pointed element is facing the external surface of the device 1, and a use configuration, in which the pointed element is facing in a direction opposite to the external surface of the device 1. In one preferred embodiment, the puncture means 2 can comprise a plurality of needles having sizes in the order of micrometers, otherwise referred to as “microneedles”. Usefully, the microneedles can be placed with respect to each other to form a matrix of pointed elements suitable to take a blood sample from the patient. The microneedles so arranged allow to perform multiple skin pricking, so as to facilitate the extraction of the blood sample.

When the device according to the present description directly comprises the puncture means 2 for obtaining the blood sample, the latter, once obtained, gathers at the abovementioned means 2 and is then conveyed into a collection chamber 3 internal to the device 1 by suitable fluidic connection means or means for conveying 4. Such a conveying can occur by blood sample suction. In particular, the suction is carried out by the means for conveying 4, having a respective suction mouth at the puncture means 2. In particular, the means for conveying 4 may be of a silicone diaphragm pump, or vacuum micropump type. Preferably, the diaphragm pump can have the following dimensions:

height equal to 0.6 mm;

width equal to 5 mm;

length equal to 5 mm.

Thus, the diaphragm pump is sufficiently small to be easily moved, and the device is scarcely bulky, light-weight and easy to carry.

The means for conveying 4 can further comprise a collection duct or cannula, e.g. a rubber catheter, in which the sample can be contacted with an anticoagulant.

Alternatively, the sample, independently extracted by the device described herein, can be directly inlet into the collection chamber, preferably after treatment with an anticoagulant.

In one alternative embodiment, the sample is introduced or conveyed into the collection chamber 3, before being contacted with an anticoagulant.

Preferably, the device 1 is provided with isolation means 14, 5, 6, 19 of polymorphonuclears (PBMCs) from the blood sample.

According to the invention, the isolation means 14, 5, 6, 19 can comprise mixing means 14, preferably inserted into the collection chamber 3, configured to mix said blood sample with a component or a group of components apt to isolate PBMCs (also defined herein as “isolation means of PBMC”). For instance, said group of components can comprise a component apt to bind to polymorphonuclears (PBMCs) present in said sample, and where necessary (i.e., when said blood sample has not yet been treated with anticoagulant), with an anticoagulating substance.

In the implementation of the device described herein, said component may be, e.g., represented by suitable microbeads onto which there are bonded antibodies against CD3+T, PBMCs-specific antigens which therefore bind PBMCs (e.g., PluriBead® or Dynabeads®-type technology).

The device 1 can further comprise a first, s1, and/or a second, s2, reservoir in which there are held respectively an anticoagulant and the component able to bind polymorphonuclear cells (polymorphonuclears), each reservoir s1, s2 being preferably provided with respective delivery means 15.

The isolation means 14, 5, 6, 19, moreover, can comprise moving means 5 and filtering means 6, described hereinafter.

Thanks to the mixing, PBMCs bind to the abovementioned component. The obtained mixture is subjected to a mechanical vibration and/or rotation stress, carried out by the action of dedicated moving means 5, preferably comprised into the collection chamber 3. In particular, the moving means 5 can be implemented by at least one rotary and/or vibrating conveyor belt onto which the mixture so obtained is conveyed.

The stress, or the stirring of the mixture by the means 5 is operated for a time of a predetermined duration, which can be a time of from 5 to 20 minutes, from 5 to 15 minutes, e.g. equal to about 10 minutes. Time duration can be measured, e.g., by a timer T preferably connected to the same moving means 5. Subsequently, the mixture is conveyed to the filtering means 6, preferably by the same moving means 5, even more preferably when the latter is implemented by a conveyor belt.

According to preferred variant embodiments, the filtering means 6 can be integrated into the moving means 5, e.g. in the form of a filtering conveyor belt. In other words, the operating surface of the conveyor belt, onto which the mixture to be conveyed is placed, can be made of filtering material. The filtering means 6 is configured to retain the PBMCs-bound component and let elute the remainder of the mixture, in fact leading to the isolation of the PBMCs bound to the abovementioned component. To this end, the filtering means 6 (belonging to the group of components apt to isolate PBMCs) has transit ports of a predetermined section, such as to prevent transit to the PBMCs-bound component and instead allow transit to the remainder of the mixture.

The selection of the cutoff of the filtering means can be easily carried out by the technician in the field depending on the size of the PBMCs-bound component. The filter cutoff should anyhow be selected in a way such as to let elute the various blood sample cells and components not bound to the abovementioned component and retain the component-PBMC complex. In one embodiment, when microbeads conjugated with PBMC-specific anti-antigen antibody are used, the filtering means will have a cutoff selected on the basis of bead diameter. E.g., for beads having a diameter of about 30 μm, the filtering means will have a cutoff such as to retain the cell-bound beads and let elute all that has a lower diameter. Wishing to use reagents already available on the market, the filtering means 6 can be implemented by pluriStrainer®-type technology, as currently available on the market.

Usefully, the isolation means 14, 5, 6, 19 can comprise separation means 19 of the polymorphonuclears (PBMCs) from the component to which they are bonded, configured to favor the separation between said polymorphonuclears (PBMCs) and said component.

Advantageously, the separation means 19 can be of the filtering membrane or buffer type.

The component-PBMC compound, retained onto the filtering means 6, are treated with the means for isolating the sole PBMCs, or separation means 19. The isolation or separation of PBMCs from the component binding them can be performed, e.g., employing a specific buffer (detachment buffer), apt to favor the breaking of the bond between the PBMCs and the component to which they are bound, the device could therefore comprise delivery means of a suitable solution for PBMCs detachment from the component to which they are bound.

The PBMCs so isolated are conveyed by collection means 9, comprising e.g. a tube or a catheter, to an analysis chamber 11. Said analysis chamber 11 preferably comprises means for the exposure of PBMCs cytoplasm, e.g., a lysis buffer for cell membranes. According to a variant embodiment, cytoplasm exposure is performed directly into the collection chamber 3.

Alternatively to what has been described hereto, in order to obtain PBMCs separation in the blood sample the group of components apt isolate PBMCs can be comprised of a micro-electrical-mechanical system allowing to have a repelling action on PBMCs and therefore to isolate them (Avivabio®) or a microfiltration Parylene membrane. Said membrane can have a filtering surface of up to 36 mm² and a porosity of about 7%-15%, with pores of different geometry (e.g., circular, oval and rectangular) having critical dimensions (sizes) that are reduced down to a few micrometers. Again, PBMCs can be separated in the blood sample by Nickel electroplating technology or filtering through paper or plastics material sheets.

After isolating the PBMCs, these are treated so as to measure the Plin2 and/or Pnpla3 and/or Rab14 protein concentration, with means making the protein presence visible, and quantifiable, at one or more predetermined wavelengths, such as, e.g. dye-conjugated, in particular fluorescent dye-conjugated detection systems. This can be obtained by treatment means 16, preferably comprised into the analysis chamber 11 and configured to carry out a bonding of the Plin2 and/or Pnpla3 and/or Rab14 protein to a component exhibiting said property, or signaling component. In one embodiment, the PBMCs sample is treated with a buffer for cytoplasm Plin2 lysis, preferably reacted with anti-Plin2 monoclonal antibodies bound to a reagent, fluorescent at predetermined wavelengths, e.g. fluorescent secondary antibodies, such as Alexa Fluor® 488. Thus, the fluorescent secondary antibodies are associated with Plin2. The device 1 can further comprise a third reservoir s3 in which the antibodies are held, provided with respective delivery means 15 of said antibodies. Furthermore, the device 1 comprises a spectrometer housed into the analysis chamber 11. Preferably, the spectrometer is provided with one radiation source 12 and of means 13 for acquiring images, both e.g. housed within the analysis chamber 11 (or the collection chamber 3), facing a deposition surface for isolated and treated PBMCs. The source 12 emits radiations at said deposition surface, said radiations having a predetermined wavelength in a manner such that the fluorescent dye-conjugated antibodies be visible (or assume a particular coloring), therefore in a manner such as to be able to single out the Plin2 by a visual inspection. In one embodiment, the radiation source 12 is configured to emit radiations at a wavelength of from 340 nm to 780 nm. Preferably, the radiation source can emit radiations at a wavelength of 488 nm. The source 12 can comprise a laser microfluorimeter or other similar devices available on the market.

Advantageously, the spectrometer 11 can have the following dimensions:

height of from 15 mm to 25 mm, preferably equal to 20.1 mm;

width of from 7 mm to 18 mm, preferably equal to 12.5 mm;

depth of from 5 mm to 15 mm, preferably equal to 10.1 mm.

Thus, the encumbrance of the spectrometer 11 is reasonable and the device 1 will be easy to carry.

The person skilled in the art will know the wavelength needed to detect the fluorescent dye of interest on the basis of simple information in the literature. The device can anyhow be made so as to be able to emit various wavelengths and therefore enable the detection of various dyes, hence without therefore binding said device to a particular and specific fluorescent dye.

Once the radiation source 12 is activated, the means 13 for acquiring images are operated to acquire images of the irradiated sample, in which the Plin2 and/or Pnpla3 and/or Rab14 protein is detectable and, above all, can be quantitated (quantified).

The device 1 preferably comprises a control unit 7, connected to all of the above-described means and components, programmed so as to control and synchronize their actuation according to an automatic mode, according to predetermined operating parameters. Said operating parameters can comprise the duration of the activation of each of the above-described means/components, as well as the blood sample pressure and/or suction rate, the conveying (conveyance) rate thereof, the amount of anticoagulant/isolation component of PBMCs/fluorescent reagent/antiPlin2 antibodies to be delivered from the respective reservoirs s1, s2, s3, and/or the wavelengths of the radiation source. Again, the control unit 7 can encompass or be connected to interface means 8, configured to allow the user to control the activation of the device 1 and/or modify/select the abovementioned operating parameters. Said interface means 8 can comprise at least one power-up button of the device 1.

Furthermore, the control unit 7 can comprise an image-processing microprocessor, programmed to identify the presence of Plin2 and/or Pnpla3 and/or Rab14 and quantitate the protein in the images acquired by the means 13. Again, the microprocessor can be programmed to quantitate the Plin2 and/or Pnpla3 and/or Rab14 protein concentration detected in the acquired images and output said data. Output data can be transmitted to external electronic devices, e.g. by wired or wireless connection means encompassed in the device 1 itself, or to display means that can be integrated in the abovementioned interface means 8, e.g., comprising a display.

The device 1 of the invention, in particular the microprocessor of the control unit 7, can be further preferably programmed for automatically comparing the datum of the Plin2 protein concentration obtained by analysis of the images acquired by the means 13 with a reference datum of the Plin2 and/or Pnpla3 and/or Rab14 protein concentration, which can be the Plin2 and/or Pnpla3 and/or Rab14 associated with a healthy subject (for simplicity's sake, hereinafter: reference datum). In this case, the device has an exclusively diagnostic function.

The abovementioned reference datum can also be stored in a memory module, e.g. encompassed in the central unit 7 and connected to the microprocessor, in which also Plin2 and/or Pnpla3 and/or Rab14 protein concentration data can be stored, detected beforehand by the device itself, on the same patient on which the analysis is being performed, in order to keep track of the evolution of the disease or to assess the effectiveness of a therapeutic treatment.

In particular, the interface means can be configured to display a graph in which the pattern of the value of Plin2 and/or Pnpla3 and/or Rab14 concentration is shown, detected as a function of time.

Furthermore, the interface means 8 can be configured to automatically display visual signals according to a predetermined color code, and/or to reproduce acoustic signals, according to the outcome of the comparison of the datum of the Plin2 protein and/or Pnpla3 and/or Rab14 concentration obtained by analysis with the reference datum.

For instance, with reference to Plin2 protein; when a monitoring of disease progress is carried out if the datum of the Plin2 protein concentration obtained from the sample analysis is lower than the reference datum, an improvement of the NAFLD is detected, which can correspond to the switching on of a green light. Instead, if the datum of the Plin2 protein concentration obtained from the sample analysis is greater than the reference datum, a deterioration of the NAFLD is detected, which can correspond to the switching on of a red light and/or to the reproduction of an acoustic alarm. Again, if the datum of the Plin2 protein concentration obtained from the sample analysis is equal to the reference datum, a stasis of the disease is detected, which can correspond to the switching on of a yellow light.

In case of diagnostic use, it is possible to use an analogous system.

Furthermore, it is possible to modify and/or select a different reference datum, and/or modify the light/acoustic signal display/automatic reproduction settings, by the same interface means 8.

Preferably, the device 1 is implemented so as to be portable, in order to allow the measurement of the Plin2 and/or Pnpla3 and/or Rab14 protein concentration also in sites distributed with respect to hospitals or medical centers. In particular, the device 1 can comprise its own powering means, allowing it to be standalone, such as, e.g., rechargeable batteries and/or photovoltaic or wind recharge devices.

The contrived device entails numerous advantages linked to the sizes of the various components, in particular to the sizes of the puncture means 2, of the spectrometer and of the pump 4. The combination of said components so sized allows the portable device 1 to be easily carried and used, as light-weight and of reduced encumbrance.

It will be understood how the portable device according to what described can be understood as suitable to allow, besides the measurement of the Plin2 protein concentration, also the measurement of the concentration of other protein types in serum/plasma or PBMC, with suitable adaptations and necessary modifications that are within the reach of a person skilled in the art.

In this regard, the device 1 can comprise connection units, preferably “plug-in” units, configured to adapt the device to other biomarkers.

According to an alternative embodiment, the device can comprise a plurality of analysis chambers 11 separated therebetween, each chamber being configured to measure the concentration in the blood sample of a different protein selected among the following: Plin2, Pnpla3 and Rab14.

Thus, the device is multiuse, being able to determine the concentration of different proteins and, therefore, allowing to determine the occurrence and the grade of different pathologies, as described in the foregoing.

Preferably, the device can comprise selection means operatively connected to the control unit 7 and configured to send the blood sample to a correct analysis chamber 11, selected based on the protein concentration to be determined. The selection means can be controlled by the control unit 7, the latter configured to send an input data containing information on the protein concentration to be determined. On the basis of the abovementioned input data, the selection means deviate the blood sample into a certain analysis chamber 11.

Advantageously, the device can comprise at least one processing unit configured to implement a computer program according to one or more of the examples reported hereinafter.

Usefully, the processing unit can be operatively connected to the control unit 7. Thus, the device can use the data detected in the analysis chambers 11 to implement the computer program(s) described hereinafter.

The device of the invention could be implemented with a suitable computer program, e.g. as defined hereinafter according to the intended use and to the method one wishes to carry out among those described and claimed herein.

Object of the invention are therefore the computer programs listed hereinafter:

A computer program for the diagnosis of NAFLD or of NASH in an individual to be analyzed, comprising a list of instructions which, when performed on an electronic computer, provided a first concentration value of the Plin2 protein in a blood sample of said individual to be analyzed or in a sample of leukocytes extracted from said blood sample and a second value of C0 and a third value of C1 as defined in the present description of protein concentration, implement the following steps:

comparing said first value with said second and third value, and

diagnosing NAFLD when said first value is greater than or equal to said value C0 and lower than said value C1, or diagnosing NASH when said first value in said blood sample is greater than or equal to said value C1.

computer program for the diagnosis of the NAS score in an individual to be analyzed, comprising a list of instructions which, when performed on an electronic computer, provided a first concentration value of the Plin2 protein in a blood sample of said individual to be analyzed or in a sample of leukocytes extracted from said blood sample, a second value C3, a third value C4 and a fourth value C5 of concentration of the Plin2 protein as defined in the present description implement the following steps:

comparing said first value with said second, third, fourth value and diagnosing the NAS score as

NAS score 1 when said first value is greater than or equal to said cutoff value C2 and lower than or equal to said cutoff value C3,

NAS score 2 when said first value is greater than said value C2 and lower than or equal to said cutoff value C4,

NAS score 3 when said first value is greater than said value C4.

computer program for the diagnosis of NASH severity in an individual to be analyzed, comprising a list of instructions which, when performed on an electronic computer, provided a first concentration value of the Plin2 protein in a blood sample of said individual to be analyzed or in a sample of leukocytes extracted from said blood sample, a second value C5, a third value C6, a fourth value C7 of concentration of the Plin2 protein as defined in the present description, implement the following steps:

comparing said first value with said second, third, fourth value and diagnosing the severity grade of NASH as

mild when said first value is greater than said value C5 and lower than said value C6,

moderate when said first value is greater than said value C6 and lower than said value C7, and

severe when said first value is greater than said value C7.

computer program for the diagnosis hepatic fibrosis in an individual to be analyzed, comprising a list of instructions which, when performed on an electronic computer, provided a first concentration value of Pnpla3 and Rab14 proteins in a blood sample of said individual to be analyzed or in a sample of leukocytes extracted from said blood sample, and a second concentration value, C8, of Pnpla3 and Rab14 proteins as defined in claim 11, implement the following steps:

comparing said first value to said second value, and

diagnosing hepatic fibrosis when said first value is greater than C8.

computer program for the diagnosis for the diagnosis of the stage of hepatic fibrosis in an individual to be analyzed, comprising a list of instructions which, when performed on an electronic computer, provided a first concentration value of Pnpla3 and Rab14 proteins in a blood sample of said individual to be analyzed or in a sample of leukocytes extracted from said blood sample, a second value C9, a third value C10 and a fourth value C11 of concentration of Pnpla3 and Rab14 proteins as defined in the present description, implement the following steps:

comparing said first value with said second, third, fourth value and diagnosing the fibrosis score stage as

stage 1 when said first value is greater than or equal to said value C9 and lower than said value C10,

stage 2 when said first value is greater than or equal to said value C10 and lower than or equal to said value C11, and

stage 3 when said first value is greater than said value C11.

Alternatively, the above-described computer programs for the diagnosis of NAFLD, NAS score, NASH, the severity grade di NASH, can comprise one of the two above-described instruction lists for the diagnosis of hepatic fibrosis and/or of the hepatic fibrosis stage.

Object of the invention are also a computer program for monitoring the effectiveness of a therapeutic treatment of NAFLD or of NASH on a patient, comprising a list of instructions which, when performed on an electronic computer, provided a first concentration value of the Plin2 protein in a blood sample of said patient or in a sample of leukocytes extracted from said blood sample at an instant of time t0 and a second concentration value of the Plin2 protein in a blood sample of said patient or in a sample of leukocytes extracted from said blood sample at an instant of time next to t0, implement the following steps:

comparing said first value to said second value, and

detecting an effectiveness of said therapeutic treatment if said second value is lower than said first value;

and a computer program for monitoring the progression of NAFLD or NASH on a patient, comprising a list of instructions which, when performed on an electronic computer, provided a first concentration value of the Plin2 protein in a blood sample of said patient or in a sample of leukocytes extracted from said blood sample at an instant of time t0 and a second concentration value of the Plin2 protein in a blood sample of said patient or in a sample of leukocytes extracted from said blood sample at an instant of time next to t0, implement the following steps:

comparing said first value to said second value, and

detecting an improvement of the NAFLD or of NASH if said second value is lower than said first value,

detecting a deterioration of the NAFLD or of NASH if said second value is greater than said first value,

detecting a stasis of the NAFLD or of NASH if said second value is approximately equal to said first value.

Object of the invention is the device in any embodiment described above and claimed, also comprising at least one processing unit configured to implement a computer program according to one or more of the above-described examples.

Usefully, the processing unit can be operatively connected to the control unit 7. Thus, the device can use the data detected in the analysis chambers 11 to implement the above-described computer program(s).

Object of the invention is also the use of the device in any one of the above-described embodiments to implement the methods object of the present invention.

Lastly, object of the invention is a therapeutic method for the treatment of patients suffering from NAFLD on which the monitoring described herein is performed.

### Examples

### Methods

19 obese subjects of both sexes suffering from NAFLD (10 women and 9 men, of average age 38.5±1.9 years and BMI 36.79±4.43 kg/m²) were enrolled and underwent bariatric surgery. A collection of peripheral venous blood with EDTA addition was performed after 12 hours of nighttime fasting at the time of enrollment. Patients had signed an informed consent to the study and surgical intervention.

Anamnesis was gathered for all subjects. An objective examination including height, weight and anthropometric measurements was also carried out. Body mass index (BMI) was computed dividing weight (kg) by height (m²). A detailed list of drugs used was gathered.

The exclusion criteria were: (1) regular and/or excessive alcohol uptake (>20 g alcohol/day for women and >30 g alcohol/day for men); (2) clinical evidence of NAFLD secondary to iatrogenic gastrointestinal or immunodeficiency (HIV infection) diseases; (3) clinical evidence of non-NAFLD hepatic diseases, including hepatitis B or C, or hemochromatosis, (4) Wilson disease, (5) glycogenosis, (6) Alpha-1 antitrypsin deficiency, (7) autoimmune hepatitis, (8) cholestasis liver disease, (9) presence of relevant cardiovascular, gastrointestinal or respiratory diseases, or any hormonal disorder, (10) clinical evidence of decompensated liver disease (Child-Pugh score >7 points), (11) undergoing narcotics abuse, (12) relevant systemic diseases (13) pregnancy.

Liver biopsies were obtained during surgery.

All subjects underwent oral glucose tolerance test (OGTT) with collections at 0, 30, 60, 90, 120, 150 and 180 minutes to measure blood insulin and sugar level (glycemia). Insulin sensitivity was measured as OGIS (acronym for Oral Glucose Insulin Sensitivity) which is a method enabling to compute insulin sensitivity from an OGTT. OGIS yields an index which is analogous to the insulin sensitivity index obtained with the clamp (Mari A, Pacini G, Brazzale A R, Ahrén R. Comparative evaluation of simple insulin sensitivity methods based on the oral glucose tolerance test. Diabetologia 2005; 48:748-751).

### Liver Histology

Fragments of liver biopsies placed under formalin were thoroughly washed with 60% isopropanol that was then vaporized. An Oil Red O (ORO) solution was added for 10 minutes, then removed, and the samples washed 4 times in water. Upon Oil Red O removal, the pieces were incubated into 100% isopropanol. Monocytes were also subjected to the same ORO staining.

Samples were then observed under an LSM 510 confocal microscope fitted with an appropriate filter.

Moreover, a part of the bioptic material obtained during surgery was mounted onto storage glass slides prepared from pieces fixed in 10% formalin, included into paraffin blocks and stained with hematoxylin and eosin in order to assess steatosis percentage. Glass slide reading was performed by an anatomic pathologist expert in liver, under blind conditions.

Brunt classification (Brunt E M, Janney C G, Di Bisceglie A M, et al. Nonalcoholic steatohepatitis: a proposal for grading and staging the histological lesions. Am J Gastroenterol. 1999; 94:2467-2474) was used to assess the histological scoring and the NAFLD/NASH (non-alcoholic steato-hepatitis) stage. In particular, steatosis was defined with the following scores: 0, absence (<1%); 1, 1%-25%; 2, 26%-50%; 3, 51%-75%; and 4, >75% of fat in the lobules. Inflammation received the following scores: 1, mild (lymphocytes isolated (scattered) or aggregated into small formations inside the portal tract and in the lobules); 2, moderate (as in grade 1, yet with greater portal and lobular infiltration); and 3, severe (same as in grade 2, yet with a more intense inflammation). The fibrosis was staged as 0 when absent, 1 when centrilobular pericellular; 2, when periportal and pericellular; 3, bridging fibrosis; and 4, cirrhosis.

### Monocyte Isolation

PBMCs were obtained from whole blood by standard gradient centrifugation on Ficoll-Hypaque (GE Healthcare Bio-Sciences, Piscataway, N.J.). The PBMCs were then washed, and the monocytes isolated by Pan Monocyte Isolation Kit.

### Hepatocyte Isolation

Liver biopsy fragments were minced and washed in HBSS to remove blood traces.

The tissue was then transferred into 50 ml test tubes containing EGTA buffer (HBSS, 0.5 mM EGTA, 0.5% BSA) and stirred 10 min at 100 rpm in a bath with water maintained at 37° C. The tissue was then placed into a digestion buffer (HBSS, 0.05% collagenase IV, 0.5% BSA without fatty acids, 10 mM CaCl2) and stirred 10 min at 100 rpm into a bath with water maintained at 37° C. The supernatant was collected and filtered through a 100 μm-pore filter and the cell suspension centrifuged at 80 rpm for 5 min at 4° C. and the supernatant discarded.

### Lipid Droplets Staining

Isolated monocytes and hepatocytes were incubated 20 min with 4% formalin and stained with Nile Red (100 ng/mL). Nuclei were stained with DAPI.

Photos were taken with a Spinning Disk confocal microscope; Crest X-Light Confocal Imager (Germany) and, to analyze the images, MetaMorph Microscopy Automation & Image Analysis Software (Molecular Devices).

### Plin2 Measurement

Monocytes and liver biopsies were homogenized in a RIPA buffer containing a protease inhibitor cocktails. Homogenates were centrifuged at 13.000 rpm for 30 min at 4° C. Protein content was measured with Bradford Protein Assay (Bio-Rad Laboratories, Hercules, Calif.). Protein lysates (30 μg) were separated on 10% SDS-PAGE and transferred on PVDF membranes. The membranes erano incubate overnight with anti-Plin2 antibodies (LS-BIO, Seattle, Wash.) and anti-βActin. Quali-quantitative analysis was performed with Chemidoc XRS Image system and Image Lab 5.0 software (Bio-Rad Laboratories, Hercules, Calif.). All data were normalized for βActin levels (8H10D10).

### Statistics

Data are expressed as mean±SD when not specified otherwise. Spearman's correlation was used to measure the degree of correlation of two variables. Agreement between the two methods was measured by Bland-Altman plot. Bland-Altman results show the difference and the mean values obtained with the two methods (Plin2 levels in monocytes and in liver). The standard deviation (SD) of the differences between the two methods is the SD bias. Agreement limits were measured as mean bias, i.e. the mean of the differences between the two measurement methods±1.96 for the SD bias.

Method sensitivity and specificity were studied by Neural Network Analysis. Neural Network Analysis parameters are reported hereinafter: 2 input layers were used: Plin2 in monocytes and subjects' age. Only 1 hidden layer, containing 6 units and hyperbolic (tangent) activation function. The output Layer is the dependent variable, i.e. the NAS level. Activation function: Softmax, and error function: cross-entropy.

Neural Network Analysis provides the area under the curve (AUC) of the ROC (Receiver Operating Characteristics) curve. The ROC curve has on the X-axis the sensitivity, and on the Y-axis the number of false positives (1−specificity). The nearest to 1 the AUC, the best the predictive value of the test. Statistical analysis was carried out by SPSS version 13.

### Results

The grade of hepatic steatosis was assessed by liver biopsy and with NAFLD Activity Score (NAS) as defined above, histological method to define the grade of NAFLD, steatosis grade means of all patients examined was of 2.42±1.17 and the subjects exhibited a steatosis grade of from 1 to 4.

In order to demonstrate that ectopic fat accumulation occurs also in monocytes Nile Red was used to stain liver biopsies and monocytes (FIG. 1, Panel A). In monocytes, lipids aggregate into larger droplets.

Plin2 expression in monocytes strongly and positively correlates with Plin2 expression in the liver (R=0.84 P<0.0001). An example of Plin2 protein expression by Western blot is reported in FIG. 1, Panel B

FIG. 2 instead reports ORO staining in a liver section and in monocytes of a same subject.

Plin2 levels in the liver correlated well with the grade of hepatic steatosis (R=0.91, P<0.001) and Plin2 levels in the monocytes correlated well with the grade of hepatic steatosis (R=0.89, P<0.001).

FIG. 3 reports the Bland-Altman graph comparing the two dosage methods, i.e. Plin2 levels in monocytes and liver. The difference between the values obtained with the two measurements is reported on the Y-axis, whereas the mean is on the Y-axis. Dotted lines report the 95% of congruence bounds between the two measurements. The graph demonstrates that all points lie inside the congruence bounds, meaning that the method of the present invention is valid.

It was also found that there is a good correlation between the Plin2 levels measured in monocytes by cytofluorimetry, expressed as median fluorescence intensity (MFI, FIG. 4), and the Plin2 levels measured by Western blot (R=0.92; P<0.0001).

Plin2 levels (MFI) in PBMCs, 48.88±5.80 (SEM), demonstrate an excellent correlation (R=0.98, P=0.0004) with the levels in monocytes, 42.90±5.77 (SEM).

As proof of correlation of the severity of non-alcoholic hepatic steatosis with Plin2 levels, it was found that Plin2 levels both in liver and in monocytes inversely correlated very well with insulin sensitivity expressed as OGIS, the regression equation for monocytes was

OGIS=−44.59*Plin2+411.3

With an R² of 0.91 and a P<0.0001

OGIS is an acronym for oral glucose insulin sensitivity index, having as measurement unit ml×min⁻¹×m⁻². Plin2 was measured by western blot in units related to beta-actin reference protein.

### Analysis on Patients

91 subjects were subjected to liver biopsy on suspected NASH, whereas 21 subjects subjected to elective cholecystectomy with normal body mass index and negative liver ultrasound examination (controls) underwent liver core biopsy during surgery. Age range was from 18 to 67 years, 55% women and 45% men.

Histological examination highlighted various stages of NAFLD activity score (NAS).

Plin2 levels in circulating monocytes, analyzed by cytofluorimetry, were used to predict NAS.

Among the controls, some had NAS=0, others NAS=1.

### Cytofluorimetry

The monoclonal antibody against Plin2 was obtained from LS-BIO (Seattle, Wash.), the fluorophore-bonded secondary monoclonal antibody, AlexaFluor 488, from Life Technology (Carlsbad, Calif.), and the anti CD14-ECD antibody from Beckman Coulter (Brea Calif.).

Monocytes were identified by use of anti CD14-ECD antibodies, used to identify the monocyte population among all polymorphonuclears. Monocytes were fixed and permeabilized by standard technique, as reported hereinafter.

1 part of fixation/permeabilization concentrate is mixed with 3 parts of fixation/permeabilization diluent. The cell pellet (2×106 cells) is resuspended in 300 of 1× permeabilization buffer and is incubated 45 min at +4° C. It is washed with 1× phosphate saline buffer (PBS), centrifuged, and cells are resuspended in 100 μL of 1×PBS. Cells in suspension are subdivided into 2 test tubes (50 μL each).
Test tube 1: cells in suspension are stained with IgG antibody Alexa Fluor 488 (1:2000) for 20 min at room environment and in the dark. This test tube is the negative control.
Test tube 2: cells in suspension are stained with Anti-Plin2 (1 μl in 50 μl), then with IgG antibody Alexa Fluor 488 and anti-CD14-ECD antibodies (4 μl in 50 μl) for 20 min at room environment in the dark.

It is washed, centrifuged and resuspended in 500 μL of 1×PBS, then proceeding with cytofluorimetric analysis.

Hence, the monocytes were stained for Plin2 using a monoclonal antibody against Plin2 and a fluorophore-bound secondary antibody (AlexaFluor 488), binding to the anti-Plin2 antibody.

The apparatus used for cytofluorimetry was FC 500 (Beckman Coulter, Brea, Calif.) and the data analyzed with Kaluza software (Beckman Coulter, Brea, Calif.).

Plin2 detection is obtained by virtue of the fluorescent tracer which generates a signal that, picked up by the cytofluorimeter photodiodes, is translated in terms of mean fluorescence intensity (MFI).

MFI is proportional to the number of antibodies that recognize and bind the cell antigen, in our case Plin2, enabling protein quantitation (Mizrahi O., Shalom E. I., Baniyash M., Klieger Y. Quantitative flow cytometry: concerns and recommendations in clinic and research. Cytometry B Clin Cytom. 2017).

Histologically, NAFLD is defined when steatosis affects more than 5% of hepatocytes, NASH instead is defined for the presence, in addition to steatosis, of ballooning degeneration of hepatocytes of any grade, and of lobular inflammatory infiltrates, regardless of their number. NAFLD Activity Score (NAS) results from the combination of steatosis, hepatocellular ballooning degeneration and lobular inflammatory infiltrates of the hepatic tissue. From the table below it can be seen that the sum of the individual components yields a maximum score of 8.

| | Score | Amount |
|---|---|---|
| Steatosis | 0 | <5% |
| | 1 | 5-33% |
| | 2 | >33-66% |
| | 3 | >66% |
| Lobular | 0 | Absence |
| inflammation | 1 | <2 foci/200x field |
| | 2 | 2-4 foci/200 x field |
| | 3 | >4 foci/200x field |
| Hepatocellular | 0 | None |
| ballooning | 1 | Few cells with |
| degeneration | | ballooning |
| | | degeneration |
| | 2 | Many cells with |
| | | ballooning |
| | | degeneration |

(Kleiner D. E., Brunt E. M., Van Natta M., Behlinh C., Contos M. J., Cummings O. W., Ferrell L. D., Liu Y.-C., Torbenson M. S., Unalp-Arida A., Yeh M., McCullough A. J., Sanyal A. J. for the Nonalcoholic Steatohepatitis Clinical Research Network. Design and validation of a histological scoring system for nonalcoholic fatty liver disease. Hepatology 41:1313-1321, 2005.)

Plin2 (Mean Fluorescence Intensity [MFI]) levels in the various NAS stages are reported in the table hereinafter:

| | | | | 95% Confidence | | | |
|---|---|---|---|---|---|---|---|
| | | | | Interval for Mean | | | |
| NAS | Number of | Plin2 | Std. | Lower | Upper | Mini- | Maxi- |
| score | Subjects | Mean | Error | Bound | Bound | mum | mum |
| 0 | 9 | 1.0032 | .17758 | .5937 | 1.4127 | .16 | 2.13 |
| 1 | 12 | 1.4150 | .31500 | −2.5875 | 5.4175 | 1.10 | 1.73 |
| 2 | 17 | 2.2843 | .24781 | 1.6779 | 2.8907 | 1.43 | 3.20 |
| 3 | 12 | 2.7217 | .20286 | 2.2752 | 3.1682 | 2.00 | 4.60 |
| 4 | 17 | 4.0659 | .23173 | 3.5746 | 4.5571 | 2.80 | 6.20 |
| 5 | 17 | 5.2986 | .85796 | 3.1992 | 7.3979 | 2.60 | 9.90 |
| 6 | 9 | 5.9000 | .18074 | 5.4354 | 6.3646 | 5.40 | 6.50 |
| 7 | 10 | 6.5286 | .10627 | 6.2685 | 6.7886 | 6.00 | 6.80 |
| 8 | 9 | 7.1300 | .08015 | 6.9339 | 7.3261 | 6.90 | 7.50 |

The correlation between mean Plin2 levels (MFI) and NAS stages is excellent, as reported hereinafter.

The independent variable, on the X-axis is the average value of Plin2, whereas the dependent one is the NAS stages (FIG. 7).

| | R | Adjusted R | Standard Error |
|---|---|---|---|
| R | Square | Square | of Estimates |
| .993 | .985 | .983 | .298 |

Independent variable is Average_Plin2.

R2 is extremely high (0,985) with a P<0.0001 significance, indicating that the model for predicting NASH histological stage, i.e., NAS, is excellent.

ANOVA

| | Sum of | | Mean | | |
|---|---|---|---|---|---|
| | Squares | df | Square | F | Sig. |
| Regression | 41.096 | 1 | 41.096 | 461.802 | .000 |
| Residues | .623 | 7 | .089 | | |
| Total | 41.719 | 8 | | | |

Independent variable is Average_Plin2.

Plin2 ability to predict the various NAS stages was studied by Neural Network Analysis.

Neural Network Analysis parameters are reported hereinafter. 2 input layers were used: Plin2 in monocytes and subjects' age. Only 1 hidden layer, containing 6 units and hyperbolic (tangent) activation function. The output layer is the dependent variable, i.e. the NAS level. Activation function: Softmax, and error function: cross-entropy.

Network Information

| Input Layer | Factors | 1 | Plin2 Monocytes |
|---|---|---|---|
| | Covariates | 1 | AGE |
| | Number of Units^{a} | | 30 |
| | Rescaling Method for | | Adjusted |
| | Covariates | | normalized |
| Hidden Layer(s) | Number of Hidden Layers | | 1 |
| | Number of Units in Hidden | | 6 |
| | Layer 1^{a} | | |
| | Activation function | | Hyperbolic |
| | | | tangent |
| Output Layer | Dependent Variables | 1 | NAS |

| | |
|---|---|
| Number of Units | 9 |
| Activation Function | Softmax |
| Error Function | Cross-entropy |

^{a}Excluding the bias unit

The design predicted incorrect NAS values only in 6.3% of cases during its training and in 11.35% of cases during the 0.05 second test, as reported hereinafter.

Model Summary

| | | |
|---|---|---|
| Training | Cross Entropy Error | 24.771 |
| | Percent Incorrect Predictions | 6.3% |
| | Stopping Rule Used | 3 consecutive step(s) |
| | | with no decrease in error^{a} |
| Testing | Training Time | 0:00:00.05 |
| | Cross Entropy Error | 19.661 |
| | Percent Incorrect Predictions | 11.35% |

Dependent Variable: NAS
^{a}Error computations are based on the testing sample

The data demonstrate that the area under the curve (AUC) of the Receiver Operating Characteristic (ROC) is very high, ranging from a minimum of 0.974 to a maximum of 1. All stages are predictable with great sensitivity and specificity.

Area Under the Curve of ROC Analysis

| | | Area |
|---|---|---|
| NAS | 0 | 1.000 |
| | 1 | 1.000 |
| | 2 | .994 |
| | 3 | .974 |
| | 4 | .991 |
| | 5 | 1.000 |
| | 6 | 1.000 |
| | 7 | .989 |
| | 8 | 1.000 |

The importance of the Plin2 variable in the model is reported hereinafter and is of 68.3%, but when normalized reaches 100%, whereas that of the second variable used in the model, i.e., age, is of 31.7%, and, when normalized, of 46.3%.

Independent Variable Importance

| | Importance | Normalized Importance |
|---|---|---|
| Plin2 Monocytes | .683 | 100.0% |
| Age | .317 | 46.3% |

### (see FIG. 8)

Multiple comparison analysis, adjusted for repeated inferences, i.e. for multiple comparisons, demonstrates the high significance of the difference among various NAS stages, see table below.

ANOVA
Plin2 Monocytes

| | | | Sum of | | Mean | | |
|---|---|---|---|---|---|---|---|
| | | | squares | df | Square | F | Sig. |
| Between | (Combined) | | 281.239 | 8 | 35.155 | 39.584 | .000 |
| Groups | Linear | Unweighted | 217.947 | 1 | 217.947 | 245.407 | .000 |
| | Term | Weighted | 275.694 | 1 | 275.694 | 310.429 | .000 |
| | | Deviation | 5.545 | 7 | .792 | .892 | .518 |

| | | | |
|---|---|---|---|
| Within Groups | 57.727 | 65 | .888 |
| Total | 338.966 | 73 | |

Multiple comparisons
Dependent variable: Plin2 Monocytes

| | | | | | | 95% Confidence | |
|---|---|---|---|---|---|---|---|
| | | | Mean | | | Interval | |
| (I) | | (J) | difference | Std. | | Lower | Upper |
| NAS | | NAS | (I − J) | Error | Sig. | Bound | Bound |
| Tukey | 0 | 1 | −.41180 | .73670 | 1.000 | −2.7756 | 1.9520 |
| HSD | | 2 | −1.28109 | .47492 | .169 | −2.8049 | .2427 |
| | | 3 | −1.71847* | .41556 | .003 | −3.0518 | −.3851 |
| | | 4 | −3.06268* | .38848 | .000 | −4.3092 | −1.8162 |
| | | 5 | −4.29537* | .47492 | .000 | −5.8192 | −2.7716 |
| | | 6 | −4.89680* | .49669 | .000 | −6.4905 | −3.3032 |
| | | 7 | −5.52537* | .47492 | .000 | −7.0492 | −4.0016 |
| | | 8 | −6.12680* | .47492 | .000 | −7.6506 | −4.6030 |
| | 1 | 0 | .41180 | .73670 | 1.000 | −1.9520 | 2.7756 |
| | | 2 | −.86929 | .75560 | .964 | −3.2937 | 1.5551 |
| | | 3 | −1.30667 | .71977 | .672 | −3.6161 | 1.0028 |
| | | 4 | −2.65088* | .70448 | .010 | −4.9113 | −.3905 |
| | | 5 | −3.88357* | .75560 | .000 | −6.3080 | −1.4592 |
| | | 6 | −4.48500* | .76946 | .000 | −6.9539 | −2.0161 |
| | | 7 | −5.11357* | .75560 | .000 | −7.5380 | −2.6892 |
| | | 8 | −5.71500* | .75560 | .000 | −8.1394 | −3.2906 |
| | 2 | 0 | 1.28109 | .47492 | .169 | −.2427 | 2.8049 |
| | | 1 | .86929 | .75560 | .964 | −1.5551 | 3.2937 |
| | | 3 | −.43738 | .44820 | .987 | −1.8755 | 1.0007 |
| | | 4 | −1.78160* | .42322 | .002 | −3.1395 | −.4237 |
| | | 5 | −3.01429* | .50373 | .000 | −4.6305 | −1.3980 |
| | | 6 | −3.61571* | .52430 | .000 | −5.2980 | −1.9335 |
| | | 7 | −4.24429* | .50373 | .000 | −5.8605 | −2.6280 |
| | | 8 | −4.84571* | .50373 | .000 | −6.4620 | −3.2295 |
| | 3 | 0 | 1.71847* | .41556 | .003 | .3851 | 3.0518 |
| | | 1 | 1.30667 | .71977 | .672 | −1.0028 | 3.6161 |
| | | 2 | .43738 | .44820 | .987 | −1.0007 | 1.8755 |
| | | 4 | −1.34422* | .35532 | .010 | −2.4843 | −.2042 |
| | | 5 | −2.57690* | .44820 | .000 | −4.0150 | −1.1388 |
| | | 6 | −3.17833* | .47120 | .000 | −4.6902 | −1.6665 |
| | | 7 | −3.80690* | .44820 | .000 | −5.2450 | −2.3688 |
| | | 8 | −4.40833* | .44820 | .000 | −5.8464 | −2.9703 |
| | 4 | 0 | 3.06268* | .38848 | .000 | 1.8162 | 4.3092 |
| | | 1 | 2.65088* | .70448 | .010 | .3905 | 4.9113 |
| | | 2 | 1.78160* | .42322 | .002 | .4237 | 3.1395 |
| | | 3 | 1.34422* | .35532 | .010 | .2042 | 2.4843 |
| | | 5 | −1.23269 | .42322 | .105 | −2.5906 | .1252 |
| | | 6 | −1.83412* | .44750 | .004 | −3.2700 | −.3983 |
| | | 7 | −2.46269* | .42322 | .000 | −3.8206 | −1.1048 |
| | | 8 | −3.06412* | .42322 | .000 | −4.4220 | −1.7062 |
| | 5 | 0 | 4.29537* | .47492 | .000 | 2.7716 | 5.8192 |
| | | 1 | 3.88357* | .75560 | .000 | 1.4592 | 6.3080 |
| | | 2 | 3.01429* | .50373 | .000 | 1.3980 | 4.6305 |
| | | 3 | 2.57690* | .44820 | .000 | 1.1388 | 4.0150 |
| | | 4 | 1.23269 | .42322 | .105 | −.1252 | 2.5906 |
| | | 6 | −.60143 | .52430 | .964 | −2.2837 | 1.0808 |
| | | 7 | −1.23000 | .50373 | .280 | −2.8463 | .3863 |
| | | 8 | −1.83143* | .50373 | .015 | −3.4477 | −.2152 |
| | 6 | 0 | 4.89680* | .49669 | .000 | 3.3032 | 6.4905 |
| | | 1 | 4.48500* | .76946 | .000 | 2.0161 | 6.9539 |
| | | 2 | 3.61571* | .52430 | .000 | 1.9335 | 5.2980 |
| | | 3 | 3.17833* | .47120 | .000 | 1.6665 | 4.6902 |
| | | 4 | 1.83412* | .44750 | .004 | .3983 | 3.2700 |
| | | 5 | .60143 | .52430 | .964 | −1.0808 | 2.2837 |
| | | 7 | −.62857 | .52430 | .954 | −2.3108 | 1.0537 |
| | | 8 | −1.23000 | .52430 | .331 | −2.9123 | .4523 |
| | 7 | 0 | 5.52537* | .47492 | .000 | 4.0016 | 7.0492 |
| | | 1 | 5.11357* | .75560 | .000 | 2.6892 | 7.5380 |
| | | 2 | 4.24429* | .50373 | .000 | 2.6280 | 5.8605 |
| | | 3 | 3.80690* | .44820 | .000 | 2.3688 | 5.2450 |
| | | 4 | 2.46269* | .42322 | .000 | 1.1048 | 3.8206 |
| | | 5 | 1.23000 | .50373 | .280 | −.3863 | 2.8463 |
| | | 6 | .62857 | .52430 | .954 | −1.0537 | 2.3108 |
| | | 8 | −.60143 | .50373 | .955 | −2.2177 | 1.0148 |
| | 8 | 0 | 6.12680* | .47492 | .000 | 4.6030 | 7.6506 |
| | | 1 | 5.71500* | .75560 | .000 | 3.2906 | 8.1394 |
| | | 2 | 4.84571* | .50373 | .000 | 3.2295 | 6.4620 |
| | | 3 | 4.40833* | .44820 | .000 | 2.9703 | 5.8464 |
| | | 4 | 3.06412* | .42322 | .000 | 1.7062 | 4.4220 |
| | | 5 | 1.83143* | .50373 | .015 | .2152 | 3.4477 |
| | | 6 | 1.23000 | .52430 | .331 | −.4523 | 2.9123 |
| | | 7 | .60143 | .50373 | .955 | −1.0148 | 2.2177 |

*The mean difference is significant at the 0.05 level.

To sum up, Plin2 in monocytes are highly predictive of NASH severity stage (i.e., of the NAS score) and enable to replace an invasive examination such as biopsy with one on peripheral blood.

### Fibrosis

Patatin-like phospholipase domain-containing protein 3 (Pnpla3) (Sigma Aldrich SAB1401851) and Ras-related protein Rab-14 (Sigma Aldrich R0656) were dosed, always in monocytes and by cytofluorimetry, on a total of 132 subjects by histological examination of the liver and monocyte isolation.

Hereinafter, the mean values (MFI) for each stage of SAF fibrosis, ranging from F0 to F4 (Bedossa P, Poitou C, Veyrie N, Bouillot J L, Basdevant A, Paradis V, et al. Histopathological algorithm and scoring system for evaluation of liver lesions in morbidly obese patients. Hepatology 2012; 56:1751-1759).

| | | | | | 95% Confidence | | | |
|---|---|---|---|---|---|---|---|---|
| | | | | | Interval for Mean | | | |
| | SAF | | Standard | Standard | Lower | Upper | | |
| | Fibrosis | Mean | Deviation | Error | Bound | Bound | Minimum | Maximum |
| Pnpla3 | 0 | .0690 | .08975 | .02838 | .0048 | .1332 | .00 | .24 |
| Monocytes | 1 | 1.2405 | .37928 | .08277 | 1.0678 | 1.4131 | .60 | 2.12 |
| | 2 | 2.3617 | .57589 | .16625 | 1.9958 | 2.7276 | 1.50 | 3.24 |
| | 3 | 1.6990 | .98323 | .31092 | .9956 | 2.4024 | .12 | 3.10 |
| | 4 | 3.1327 | .65606 | .16940 | 2.7694 | 3.4960 | 1.96 | 3.91 |
| | Total | 1.7509 | 1.14752 | .13916 | 1.4731 | 2.0286 | .00 | 3.91 |
| Rab14 | 0 | .0000 | .00000 | .00000 | .0000 | .0000 | .00 | .00 |
| Monocytes | 1 | .0000 | .00000 | .00000 | .0000 | .0000 | .00 | .00 |
| | 2 | .0458 | .15877 | .04583 | −.0550 | .1467 | .00 | .55 |
| | 3 | .2191 | .27432 | .08271 | .0348 | .4034 | .00 | .69 |
| | 4 | .6733 | .24873 | .06422 | .5356 | .8111 | .00 | .91 |
| | Total | .1893 | .31571 | .03801 | .1134 | .2651 | .00 | .91 |

ANOVA

| | | Sum of | | Mean | | |
|---|---|---|---|---|---|---|
| | | Squares | df | Square | F | Sig. |
| Pnpla3 | Between Groups | 66.902 | 4 | 16.725 | 49.413 | .000 |
| Mono- | Within Groups | 21.324 | 63 | .338 | | |
| cytes | Total | 88.226 | 67 | | | |
| Rab14 | Between Groups | 4.882 | 4 | 1.220 | 41.200 | .000 |
| Mono- | Within Groups | 1.896 | 64 | .030 | | |
| cytes | Total | 6.778 | 68 | | | |

Then a neural network analysis was performed to compute the AUCs of ROC curves, using Pnpla3Monocytes and Rab14Monocytes as independent variables and NAS as dependent variable and the presence of absence of diabetes as covariate (binary variable, Yes=1, No=0). These variables were used in the input layer of the neural network. Only 1 hidden layer with 8 units, the activation function was a hyperbole. The output of the model yielded the grade of fibrosis (SAF F), the activation function was softmax and the error function was cross-entropy.

Case processing summary

| | N | Percent |
|---|---|---|
| Training Sample | 35 | 85.4% |
| Testing Sample | | |
| | 6 | 14.6% |
| Valid | 41 | 100.0% |
| Excluded | 91 | |
| Total | 132 | |

Network Information

| Input Layer | Factors | 1 | Pnpla3Monocytes |
|---|---|---|---|
| | | 2 | Rab14Monocytes |
| | Covariates | 1 | DIABETES |
| | | | (YES = 1; |
| | | | NO = 0) |
| | Number of Units^{a} | | 35 |
| | Rescaling Method for | | Standardized |
| | Covariates | | |
| Hidden Layer(s) | Number of Hidden Layers | | 1 |
| | Number of Units in Hidden | | 8 |
| | Layer 1^{a} | | |
| | Activation Function | | Hyperbolic |
| | | | tangent |
| Output Layer | Dependent Variables | 1 | SAF F |

| | |
|---|---|
| Number of Units | 4 |
| Activation Function | Softmax |
| Error Function | Cross-entropy |

^{a}Excluding the bias unit

c

| | | |
|---|---|---|
| Training | Cross-Entropy Error | 17.407 |
| | Percent Incorrect Predictions | 14.3% |
| | Stopping Rule Used | 1 consecutive step(s) with |
| | | no decrease in error^{a} |
| | Training Time | 0:00:00.05 |
| Testing | Cross-Entropy Error | 2.101 |
| | Percent Incorrect Predictions | 0.0% |

Dependent Variable: SAF F
^{a}Error computations are based on the testing sample.

During training of the model, the error percentage was of 14.3% and during the test of test 0%, time used was 0.05 minutes.

The Area Under the Curve (AUC) of ROC analysis is reported in the table hereinafter:

ROC AUC

| | | Area |
|---|---|---|
| SAF F | 0 | 1.000 |
| | 1 | .950 |
| | 2 | 1.000 |
| | 3 | .955 |

Hence, stage 0, i.e. absence of fibrosis, is predicted at 100%, stage 1 at 95%, 2 at 100% and 3 at 95.5%.

The importance of the independent variables (Pnpla3Monocytes and Rab14Monocytes) in the model was of 62%, but when normalized of 100%, that of Pnpla3Monocytes of 29.7%, that when normalized reaches 47.9% for Rab14Monocytes and that of the presence or absence of diabetes of 0.84%, when normalized of 13.5%.

Independent Variable Importance

| | Importance | Normalized Importance |
|---|---|---|
| Pnpla3Monocytes | .620 | 100.0% |
| Rab14Monocytes | .297 | 47.9% |
| DIABETES | .084 | 13.5% |
| (YES = 1; NO = 0) | | |

### (see FIG. 9)

Hence, the use of Pnpla3 and Rab14 enables to give very accurate information on fibrosis.

However, also Plin2 alone gives a good indication of fibrosis.

Case Processing Summary

| | | N | Percent |
|---|---|---|---|
| Sample | Training | 35 | 87.5% |
| | Testing | 5 | 12.5% |

| | | |
|---|---|---|
| Valid | 40 | 100.0% |
| Excluded | 92 | |
| Total | 132 | |

Network Information

| Input Layer | Factors | 1 | Plin2 Monocytes |
|---|---|---|---|
| | Covariates | 1 | DIABETES |
| | | | (YES = 1; |
| | | | NO = 0) |
| | Number of Units^{a} | | 30 |
| | Rescaling Method for | | Standardized |
| | Covariate | | |
| Hidden Layer(s) | Number of Hidden Layers | | 1 |
| | Number of Units in Hidden | | 16 |
| | Layer 1^{a} | | |
| | Activation Function | | Hyperbolic |
| | | | Tangent |
| Output Layer | Dependent Variables | 1 | SAF F |

| | |
|---|---|
| Number of Units | 5 |
| Activation Function | Softmax |
| Error Function | Cross-entropy |

^{a}Excluding the bias unit

Model Summary

| | | |
|---|---|---|
| Training | Cross-entropy Error | 13.463 |
| | Percent Incorrect Predictions | 14.3% |
| | Stopping Rule Used | 1 consecutive step(s) with |
| | | no decrease in error^{a} |
| | Training Time | 0:00:00.08 |
| Testing | Cross-entropy Error | 3.079 |
| | Percent Incorrect Predictions | 20.0% |

Dependent variable: SAF F
^{a}Error computations are based on the testing sample.

Percent incorrect predictions during training: 14.3%, and, during test: 20%.

The ROC AUC reported hereinafter for Plin2 in defining fibrosis is 98.9% for stage 0 of fibrosis, 94% for stage 1, 97% for stage 2, 98.8% for stage 3 and 100% for stage 4.

Area Under the Curve

| | | Area |
|---|---|---|
| SAF F | 0 | .989 |
| | 1 | .940 |
| | 2 | .970 |
| | 3 | .988 |
| | 4 | 1.000 |

The importance of Plin2 in monocytes in diagnosing fibrosis is 49.2%, and, when normalized, 96.8%, that of the presence or absence of diabetes is 50.8%, and, when normalized, 100%, as reported in the Table and in FIG. 10.

Independent Variable Importance

| | Importance | Normalized Importance |
|---|---|---|
| Plin2 Monocytes | .492 | 96.8% |
| DIABETES | .508 | 100.0% |
| (YES = 1; NO = 0) | | |

ANOVA
Plin2 Monocytes

| | | | Sum of | | Mean | | |
|---|---|---|---|---|---|---|---|
| | | | Squares | df | Square | F | Sig. |
| Between | (Combined) | | 281.239 | 8 | 35.155 | 39.584 | .000 |
| Groups | Linear | Un- | 217.947 | 1 | 217.947 | 245.407 | .000 |
| | Term | weighted | | | | | |
| | | Weighted | 275.694 | 1 | 275.694 | 310.429 | .000 |
| | | Deviation | 5.545 | 7 | .792 | .892 | .518 |

| | | | |
|---|---|---|---|
| Within Groups | 57.727 | 65 | .888 |
| Total | 338.966 | 73 | |

Multiple Comparisons
Dependent Variable: Plin2 Monocytes

| | | | | | | 95% Confidence | |
|---|---|---|---|---|---|---|---|
| | | | Mean | | | Interval | |
| (I) | | (J) | Difference | Std. | | Lower | Upper |
| NAS | | NAS | (I − J) | Error | Sig. | Bound | Bound |
| Tukey | 0 | 1 | −.41180 | .73670 | 1.000 | −2.7756 | 1.9520 |
| HSD | | 2 | −1.28109 | .47492 | .169 | −2.8049 | .2427 |
| | | 3 | −1.71847* | .41556 | .003 | −3.0518 | −.3851 |
| | | 4 | −3.06268* | .38848 | .000 | −4.3092 | −1.8162 |
| | | 5 | −4.29537* | .47492 | .000 | −5.8192 | −2.7716 |
| | | 6 | −4.89680* | .49669 | .000 | −6.4905 | −3.3032 |
| | | 7 | −5.52537* | .47492 | .000 | −7.0492 | −4.0016 |
| | | 8 | −6.12680* | .47492 | .000 | −7.6506 | −4.6030 |
| | 1 | 0 | .41180 | .73670 | 1.000 | −1.9520 | 2.7756 |
| | | 2 | −.86929 | .75560 | .964 | −3.2937 | 1.5551 |
| | | 3 | −1.30667 | .71977 | .672 | −3.6161 | 1.0028 |
| | | 4 | −2.65088* | .70448 | .010 | −4.9113 | −.3905 |
| | | 5 | −3.88357* | .75560 | .000 | −6.3080 | −1.4592 |
| | | 6 | −4.48500* | .76946 | .000 | −6.9539 | −2.0161 |
| | | 7 | −5.11357* | .75560 | .000 | −7.5380 | −2.6892 |
| | | 8 | −5.71500* | .75560 | .000 | −8.1394 | −3.2906 |
| | 2 | 0 | 1.28109 | .47492 | .169 | −.2427 | 2.8049 |
| | | 1 | .86929 | .75560 | .964 | −1.5551 | 3.2937 |
| | | 3 | −.43738 | .44820 | .987 | −1.8755 | 1.0007 |
| | | 4 | −1.78160* | .42322 | .002 | −3.1395 | −.4237 |
| | | 5 | −3.01429* | .50373 | .000 | −4.6305 | −1.3980 |
| | | 6 | −3.61571* | .52430 | .000 | −5.2980 | −1.9335 |
| | | 7 | −4.24429* | .50373 | .000 | −5.8605 | −2.6280 |
| | | 8 | −4.84571* | .50373 | .000 | −6.4620 | −3.2295 |
| | 3 | 0 | 1.71847* | .41556 | .003 | .3851 | 3.0518 |
| | | 1 | 1.30667 | .71977 | .672 | −1.0028 | 3.6161 |
| | | 2 | .43738 | .44820 | .987 | −1.0007 | 1.8755 |
| | | 4 | −1.34422* | .35532 | .010 | −2.4843 | −.2042 |
| | | 5 | −2.57690* | .44820 | .000 | −4.0150 | −1.1388 |
| | | 6 | −3.17833* | .47120 | .000 | −4.6902 | −1.6665 |
| | | 7 | −3.80690* | .44820 | .000 | −5.2450 | −2.3688 |
| | | 8 | −4.40833* | .44820 | .000 | −5.8464 | −2.9703 |
| | 4 | 0 | 3.06268* | .38848 | .000 | 1.8162 | 4.3092 |
| | | 1 | 2.65088* | .70448 | .010 | .3905 | 4.9113 |
| | | 2 | 1.78160* | .42322 | .002 | .4237 | 3.1395 |
| | | 3 | 1.34422* | .35532 | .010 | .2042 | 2.4843 |
| | | 5 | −1.23269 | .42322 | .105 | −2.5906 | .1252 |
| | | 6 | −1.83412* | .44750 | .004 | −3.2700 | −.3983 |
| | | 7 | −2.46269* | .42322 | .000 | −3.8206 | −1.1048 |
| | | 8 | −3.06412* | .42322 | .000 | −4.4220 | −1.7062 |
| | 5 | 0 | 4.29537* | .47492 | .000 | 2.7716 | 5.8192 |
| | | 1 | 3.88357* | .75560 | .000 | 1.4592 | 6.3080 |
| | | 2 | 3.01429* | .50373 | .000 | 1.3980 | 4.6305 |
| | | 3 | 2.57690* | .44820 | .000 | 1.1388 | 4.0150 |
| | | 4 | 1.23269 | .42322 | .105 | −.1252 | 2.5906 |
| | | 6 | −.60143 | .52430 | .964 | −2.2837 | 1.0808 |
| | | 7 | −1.23000 | .50373 | .280 | −2.8463 | .3863 |
| | | 8 | −1.83143* | .50373 | .015 | −3.4477 | −.2152 |
| | 6 | 0 | 4.89680* | .49669 | .000 | 3.3032 | 6.4905 |
| | | 1 | 4.48500* | .76946 | .000 | 2.0161 | 6.9539 |
| | | 2 | 3.61571* | .52430 | .000 | 1.9335 | 5.2980 |
| | | 3 | 3.17833* | .47120 | .000 | 1.6665 | 4.6902 |
| | | 4 | 1.83412* | .44750 | .004 | .3983 | 3.2700 |
| | | 5 | .60143 | .52430 | .964 | −1.0808 | 2.2837 |
| | | 7 | −.62857 | .52430 | .954 | −2.3108 | 1.0537 |
| | | 8 | −1.23000 | .52430 | .331 | −2.9123 | .4523 |
| | 7 | 0 | 5.52537* | .47492 | .000 | 4.0016 | 7.0492 |
| | | 1 | 5.11357* | .75560 | .000 | 2.6892 | 7.5380 |
| | | 2 | 4.24429* | .50373 | .000 | 2.6280 | 5.8605 |
| | | 3 | 3.80690* | .44820 | .000 | 2.3688 | 5.2450 |
| | | 4 | 2.46269* | .42322 | .000 | 1.1048 | 3.8206 |
| | | 5 | 1.23000 | .50373 | .280 | −.3863 | 2.8463 |
| | | 6 | .62857 | .52430 | .954 | −1.0537 | 2.3108 |
| | | 8 | −.60143 | .50373 | .955 | −2.2177 | 1.0148 |
| | 8 | 0 | 6.12680* | .47492 | .000 | 4.6030 | 7.6506 |
| | | 1 | 5.71500* | .75560 | .000 | 3.2906 | 8.1394 |
| | | 2 | 4.84571* | .50373 | .000 | 3.2295 | 6.4620 |
| | | 3 | 4.40833* | .44820 | .000 | 2.9703 | 5.8464 |
| | | 4 | 3.06412* | .42322 | .000 | 1.7062 | 4.4220 |
| | | 5 | 1.83143* | .50373 | .015 | .2152 | 3.4477 |
| | | 6 | 1.23000 | .52430 | .331 | −.4523 | 2.9123 |
| | | 7 | .60143 | .50373 | .955 | −1.0148 | 2.2177 |

*The mean difference is significant at the 0.05 level.

1. A method for the diagnosis of non-alcoholic steatohepatitis (NASH) comprising the steps of
a. measuring the concentration value of the Plin2 protein in a blood sample or in a sample of leukocytes extracted from said blood sample of an individual,
b. comparing the value obtained in point a. with the concentration value of the Plin2 protein in a blood sample or in a sample of leukocytes extracted from said blood sample of a healthy individual,
c. diagnosing NASH when the value measured in a. is greater than the value measured in b.

2. A method for the diagnosis of NASH, comprising the steps of
a. measuring the concentration value of the Plin2 protein in a blood sample or in a sample of leukocytes extracted from said blood sample
b. measuring the concentration value of the Plin2 protein in a population of blood samples coming from patients suffering from NASH and of healthy patients and identifying a cutoff value, C1, of said concentration between patients suffering from NASH and healthy patients,
c. comparing the value obtained in point a. with the cutoff values obtained in point b, and
d. diagnosing NASH when the value obtained in a. is greater than or equal to said value C1.

3. A method for the diagnosis of the NAS score of patients suffering from NAFLD or NASH, comprising the steps of
a. measuring the concentration value of the Plin2 protein in a blood sample or in a sample of leukocytes extracted from said blood sample
b. measuring the concentration value of the Plin2 protein in a population of blood samples coming from patients suffering from NAFLD or NASH and of healthy patients, wherein, in said population comprising samples coming from patients suffering from NAFLD, the NAS score value was histologically defined in said patients as NAS 1, NAS 2 and NAS 3, on the basis of the percentage of steatosis in liver cells, the occurrence of ballooned hepatocytes, the presence of lobular inflammation and the presence of portal inflammation and identifying:
a cutoff value, C2, of said concentration between healthy patients and patients suffering from NAFLD with NAS score 1;
a cutoff value, C3, of said concentration between patients suffering from NAFLD with NAS 1 and patients suffering from NAFLD with NAS score 2; and
a cutoff value, C4, of said concentration between patients suffering from NAFLD with NAS 2 and patients suffering from NASH with NAS score 3,
c. comparing the value obtained in point a. with the cutoff values obtained in point b
d. diagnosing the NAS score as
NAS score 1 when the value obtained in a. is greater than or equal to said cutoff value C2 and lower than or equal to said cutoff value C3,
NAS score 2 when the value obtained in a. is greater than said value C2 and lower than or equal to said cutoff value C4,
NAS score 3 when the value obtained in a. is greater than said value C4.

4. A method for the diagnosis of the severity grade of NASH, comprising the steps of
a. measuring the concentration value of the Plin2 protein in a blood sample or in a sample of leukocytes extracted from said blood sample
b. measuring the concentration value of the Plin2 protein in a population of blood samples coming from patients suffering from NASH and of healthy patients wherein, in said population comprising samples coming from patients suffering from NASH, the severity grade of the pathology was histologically defined in said patients as mild, moderate or severe on the basis of the percentage of steatosis in liver cells, the occurrence of ballooned hepatocytes, the presence of lobular inflammation and the presence of portal inflammation and identifying
a cutoff value, C5, of said concentration between healthy patients and patients suffering from mild NASH;
a cutoff value, C6, of said concentration between patients suffering from mild NASH and patients suffering from moderate NASH; and
a cutoff value, C7, of said concentration between patients suffering from moderate NASH and patients suffering from severe NASH,
c. comparing the value obtained in point a. with the cutoff values obtained in point b
d. diagnosing the severity grade of NASH as
mild when the value obtained in a. is greater than said value C5 and lower than said value C6,
moderate, when the value obtained in a. is greater than said value C6 and lower than said value C7, and
severe, when the value obtained in a. is greater than said value C7.

5. The method according to claim 1, wherein said concentration value of the Plin2 protein is measured by Western blot, or Cytofluorimetry, or ELISA, or quantitative PCR, preferably wherein said concentration value of the Plin2 protein is measured by cytofluorimetry using a monoclonal antibody specifically binding Plin2 and not binding other proteins, labeled with a suitable fluorochrome, and said cutoff is expressed in terms of mean fluorescence intensity (MFI).

6. (canceled)

7. The method for the diagnosis of NASH, comprising the steps of
a. measuring the concentration value of the Plin2 protein in a blood sample or in a sample of leukocytes extracted from said blood sample, wherein said value is measured by cytofluorimetry using a monoclonal antibody specifically binding Plin2 and not binding other proteins labeled with a suitable fluorochrome
b. comparing the value obtained in point a. with a cutoff value of said concentration expressed in terms of mean fluorescence intensity (MFI) equal to 2.7 MFI e with a cutoff value of said concentration equal to 1.0 MFI
d. diagnosing NASH when the value obtained in a. is greater than 2.7 MFI.

8. A method for the diagnosis of the NAS score of patients suffering from NAFLD or NASH, comprising the steps of
a. measuring the concentration value of the Plin2 protein in a blood sample or in a sample of leukocytes extracted from said blood sample in said value is measured by cytofluorimetry using a monoclonal antibody specifically binding Plin2 and not binding other proteins labeled with a suitable fluorochrome,
c. comparing the value obtained in point a. with cutoff values of said concentration expressed in terms of mean fluorescence intensity (MFI) equal to 1.0 MFI; 1.4 MFI and 2.7 MFI
d. diagnosing NAS score 1 when the value obtained in a. is greater than or equal to the cutoff value of 1.0 MFI and lower than or equal to the cutoff value of 1.4 MFI; diagnosing NAS score 2 when the value obtained in a. is greater than the cutoff value of 1.4 MFI, or diagnosing NAS score 3 when the value obtained in a. is greater than the cutoff value of 2.7 MFI.

9. A method for the diagnosis of the severity grade of NASH, comprising the steps of
a. measuring the concentration value of the Plin2 protein in a blood sample or in a sample of leukocytes extracted from said blood sample in said value is measured by cytofluorimetry using a monoclonal antibody specifically binding Plin2 and not binding other proteins labeled with a suitable fluorochrome,
c. comparing the value obtained in point a. with cutoff values of said concentration expressed in terms of mean fluorescence intensity (MFI) equal to 2.7 MFI, 4 MFI and 6.3 MFI
d. diagnosing NASH in mild form when the value obtained in a. is greater than the cutoff value of 2.7 MFI and lower than or equal to the cutoff value of 4 MFI, NASH in moderate form when the value obtained in a. is greater than the cutoff value of 4 MFI and lower than or equal to the value of 6.3 MFI, and NASH in severe form when the value obtained in a. is greater than 6.3 MFI.

10. (canceled)

11. The method according to claim 2, further comprising the steps
e. measuring the concentration value of Pnpla3 and Rab14 proteins in a blood sample or in a sample of leukocytes extracted from said blood sample in point a. for which NASH was diagnosed
f. measuring the concentration value of Pnpla3 and Rab14 proteins in a population of blood samples coming from patients suffering from hepatic fibrosis and healthy patients and identifying a cutoff value, C8, of said concentration between patients suffering from hepatic fibrosis and healthy patients,
g. comparing the value obtained in point e. with the cutoff value obtained in point f
h. diagnosing hepatic fibrosis when the value obtained in point e. is greater than said value C8.

12. The method according to claim 2, further comprising the steps of
e. measuring the protein concentration value of Pnpla3 and Rab14 protein proteins in a blood sample or in a sample of leukocytes extracted from said blood sample in point a. for which NASH was diagnosed
f. measuring the concentration value of Pnpla3 and Rab14 proteins in a population of blood samples coming from healthy patients and from patients suffering from hepatic fibrosis, wherein the severity grade of said pathology was histologically defined as stage 1, stage 2 or stage 3 on the basis of the position and extension of the hepatic fibrosis, and identifying
a cutoff value, C9, of said concentration between healthy patients and patients suffering from stage 1 hepatic fibrosis;
a cutoff value, C10, of said concentration between patients suffering from stage 1 hepatic fibrosis and patients suffering from stage 2 hepatic fibrosis; and
a cutoff value, C11, of said concentration between patients suffering from stage 2 hepatic fibrosis and patients suffering from stage 3 hepatic fibrosis,
g. comparing the value obtained in point e. with the cutoff values obtained in point f
h. diagnosing the stage of hepatic fibrosis as
stage 1 when the value obtained in point e. is greater than or equal to said value C9 and lower than said value C10,
stage 2 when the value obtained in point e. is greater than or equal to said value C10 and lower than or equal to said value C11, and
stage 3 when the value obtained in point e. is greater than said value C11.

13. The method according to claim 11, wherein said concentration value of Pnpla3 and Rab14 proteins is measured by Western blot, or Cytofluorimetry, or ELISA, or quantitative PCR, preferably wherein said concentration value of Pnpla3 and Rab14 proteins is measured by cytofluorimetry using a monoclonal antibody specifically binding Pnpla3 and not binding other proteins, and a monoclonal antibody specifically binding Rab14 and not binding other proteins, labeled with a suitable fluorochrome.

14. (canceled)

15. The method according to claim 2, further comprising the steps of
e. measuring the concentration value of Pnpla3 and Rab14 proteins in a blood sample or in a sample of leukocytes extracted from said blood sample in point a. for which NASH was diagnosed, wherein said value is measured by cytofluorimetry using a monoclonal antibody specifically binding PNPLA and not binding other proteins, and a monoclonal antibody specifically binding Rab14 and not binding other proteins, labeled with a suitable fluorochrome,
g. comparing the value obtained in point e. with a cutoff value, expressed in terms of mean fluorescence intensity (MFI) greater than or equal to 1.24 WI
h. diagnosing hepatic fibrosis when the value obtained in point e. is greater than or equal to 1.24 MFI.

16. The method according to claim 2 further comprising the steps of
e. measuring the concentration value of Pnpla3 and Rab14 proteins in a blood sample or in a sample of leukocytes extracted from said blood sample in point a. for which NASH was diagnosed, wherein said value is measured by cytofluorimetry using a monoclonal antibody specifically binding PNPLA and not binding other proteins, and a monoclonal antibody specifically binding Rab14 and not binding other proteins, labeled with a suitable fluorochrome, and said cutoff is expressed in terms of mean fluorescence intensity (MFI),
g. comparing the value obtained in point a. with cutoff values of said concentration expressed in terms of mean fluorescence intensity (MFI) equal to 1.24 MFI, 2.3 MFI and 3.10 MFI
h. diagnosing mild stage 1 hepatic fibrosis when the value obtained in point e. is greater than or equal to 1.24 MFI and lower than 2.4 MFI, stage 2 fibrosis when the value obtained in point e. is greater than or equal to 2.4 MFI and lower than 3.10 MFI, and stage 3 hepatic fibrosis when the value obtained in point e. is greater than or equal to 3.10 MFI.

17. (canceled)

18. A method for monitoring the effectiveness of a therapeutic treatment of NASH on a patient, comprising the steps of
a. measuring the concentration value of the Plin2 protein in a blood sample or in a sample of leukocytes extracted from said blood sample of an individual suffering from NASH at an instant of time t0 from which said monitoring is carried out
b. measuring the concentration value of the Plin2 protein in a blood sample or in a sample of leukocytes extracted from said blood sample of an individual suffering from NASH at one or more time instants tn, wherein n is an integer greater than 0, and in which each tn corresponds to instants of time following t0, wherein
a decrease in the concentration of the Plin2 protein in one or more of said time instants tn is indicative of an effectiveness of said therapeutic treatment.

19. A method for monitoring the progression of NASH on a patient, comprising the steps of
a. measuring the concentration value of the Plin2 protein in a blood sample or in a sample of leukocytes extracted from said blood sample of an individual suffering from NASH at an instant of time t0 from which said monitoring is carried out
b. measuring the concentration value of the Plin2 protein in a blood sample or in a sample of leukocytes extracted from said blood sample of an individual suffering from NASH at one or more time instants tn, wherein n is an integer greater than 0, and in which each tn corresponds to instants of time following t0, wherein
a decrease in the concentration of the Plin2 protein in one or more of said time instants tn is indicative of an improvement of the NASH, whereas an increase in the concentration of the Plin2 protein in one or more of said time instants tn is indicative of the deterioration of the NASH.

20. The method according to claim 18, wherein said concentration value of the Plin2 protein is measured by Western blot, or Cytofluorimetry, or ELISA, or quantitative PCR, preferably wherein said concentration value of Pnpla3 and Rab14 proteins is measured by cytofluorimetry using a monoclonal antibody specifically binding Pnpla3 and not binding other proteins, and a monoclonal antibody specifically binding Rab14 and not binding other proteins, labeled with a suitable fluorochrome.

21. (canceled)

22. (canceled)

23. The method according to claim 1, wherein said blood sample is a peripheral blood sample and/or said leukocytes are polymorphonuclear and/or monocyte cells.

24. (canceled)

25. A computer program for the diagnosis of NASH in an individual to be analyzed, comprising a list of instructions which, when performed on an electronic computer, provided a first concentration value of the Plin2 protein in a blood sample of said individual to be analyzed or in a sample of leukocytes extracted from said blood sample and a second concentration value of the Plin2 protein in a blood sample or in a sample of leukocytes extracted from said blood sample of a healthy individual, implement the following steps:
comparing said first value to said second value, and
diagnosing NASH when said first value is greater than said second value.

26. A computer program for the diagnosis of NASH in an individual to be analyzed, comprising a list of instructions which, when performed on an electronic computer, provided a first concentration value of the Plin2 protein in a blood sample of said individual to be analyzed or in a sample of leukocytes extracted from said blood sample and a second value of C0 and a third value of C1 as defined in claim 2 of protein concentration, implement the following steps:
comparing said first value with said second and third value, and
diagnosing NASH when said first value in said blood sample is greater than or equal to said value C1.

27. (canceled)

28. A computer program for the diagnosis of NASH severity in an individual to be analyzed, comprising a list of instructions which, when performed on an electronic computer, provided a first concentration value of the Plin2 protein in a blood sample of said individual to be analyzed or in a sample of leukocytes extracted from said blood sample, a second value C5, a third value C6, a fourth value C7 of concentration of the Plin2 protein as defined in claim 4, implement the following steps:
comparing said first value with said second, third, fourth value and diagnosing the severity grade of NASH as
mild when said first value is greater than said value C5 and lower than said value C6,
moderate when said first value is greater than said value C6 and lower than said value C7, and
severe when said first value is greater than said value C7.

29. (canceled)

30. (canceled)

31. A computer program for monitoring the effectiveness of a therapeutic treatment of NAFLD or of NASH on a patient, comprising a list of instructions which, when performed on an electronic computer, provided a first concentration value of the Plin2 protein in a blood sample of said patient or in a sample of leukocytes extracted from said blood sample at an instant of time t0 and a second concentration value of the Plin2 protein in a blood sample of said patient or in a sample of leukocytes extracted from said blood sample at an instant of time subsequent to t0, implement the following steps:
comparing said first value to said second value, and
detecting an effectiveness of said therapeutic treatment if said second value is lower than said first value.

32. A computer program for monitoring the progression of NASH on a patient, comprising a list of instructions which, when performed on an electronic computer, provided a first concentration value of the Plin2 protein in a blood sample of said patient or in a sample of leukocytes extracted from said blood sample at an instant of time t0 and a second concentration value of the Plin2 protein in a blood sample of said patient or in a sample of leukocytes extracted from said blood sample at an instant of time next to t0, implement the following steps:
comparing said first value to said second value, and
detecting an improvement of the NASH if said second value is lower than said first value,
detecting a deterioration of the NASH if said second value is greater than said first value,
detecting a stasis of the NASH if said second value is approximately equal to said first value.

33. A device (10) for automatic measurement of Plin2 and/or Pnpla3 and/or Rab14 protein concentration in a patient's blood sample, comprising:
puncture means (2) apt to pierce the skin of an individual from which said blood sample is to be taken;
isolation means (14, 5, 6, 19) of polymorphonuclears (PBMCs) in a blood sample, comprising at least separation means (19) of said polymorphonuclears (PBMCs) from the component to which they are bonded, configured to favor the separation between said polymorphonuclears (PBMCs) and said component, said separation means (19) being of the filtering membrane or buffer type;
at least one spectrometer comprising at least one analysis chamber (11) and measurement means (16, 12, 13) of the concentration of the Plin2 and/or Pnpla3 and/or Rab14 protein in the cytoplasm of said polymorphonuclears (PBMCs), of which one spectrometer (12, 13), said measurement means (16, 12, 13) comprising
treatment means (16) of the polymorphonuclears (PBMCs) isolated by said isolation means (14, 5, 6, 19), configured in such a way as to allow the binding of the Plin2 and/or Pnpla3 and/or Rab14 protein, when present, with a specific reagent for said protein, wherein said reagent is fluorescent or has a specific coloring at one or more predetermined wavelengths;
at least one spectrometer (12, 13) provided with:
one radiation source (12), configured to emit radiations at a predetermined wavelength at said isolated and treated polymorphonuclears (PBMCs), in such a manner that said reagent emits fluorescence or presents said specific coloring;
means (13) for acquiring images of the sample irradiated by the radiation source (12); and
a control unit (7) connected to said isolation means (14, 5, 6, 19) and measurement means (16, 12, 13), programmed in such a way as to:
control and synchronize the actuation of said isolation means (14, 5, 6, 19) and measurement means (16, 12, 13) according to an automatic mode, according to predetermined operating parameters,
automatically comparing the concentration data of the Plin2 and/or Pnpla3 and/or Rab14 protein measured in the cytoplasm of said polymorphonuclears (PBMCs) with at least one reference datum of Plin2 and/or Pnpla3 and/or Rab14 protein concentration,
wherein said device further comprises at least one processing unit configured to implement a computer program according to claim 18.

34.-45. (canceled)
